(19) United States
(12) Patent Application Publication
(10) Pub. No.: US 2025/0213810 A1
(43) Pub. Date: Jul. 3, 2025

(54) AIRWAY MANAGEMENT DEVICE WITH EPIGLOTTIS HOLDING FEATURES

(71) Applicant: ONY Biotech Inc., Amherst, NY (US)

(72) Inventors: Daniel Swartz, Varysburg, NY (US); Corey Commaroto, Lockport, NY (US); Kari Roberts, St. Paul, MN (US)

(73) Assignee: ONY Biotech Inc., Amherst, NY (US)

(21) Appl. No.: 19/084,600

(22) Filed: Mar. 19, 2025

Related U.S. Application Data
(63) Continuation-in-part of application No. 17/166,950, filed on Feb. 3, 2021.
Application No. 17/166,950 is a continuation-in-part of application No. 16/864,248, filed on May 1, 2020.
Application No. 16/864,248 is a continuation-in-part of application No. PCT/US2019/045830, filed on Aug. 9, 2019.

(51) Int. Cl. A61M 16/04

(52) U.S. Cl. CPC A61M 16/0434; A61M 16/042; A61M 16/0431; A61M 2202/0007; A61M 2202/02; A61M 2205/0216; A61M 2210/065; A61M 2210/0656

(57) ABSTRACT

An airway management device (“AMD”) is described. Such an AMD may have a laryngeal mask, a portion of which is configured to fit against a patient at an area surrounding the patient's laryngeal inlet, and thereby cover a laryngeal inlet of the patient. Some embodiments of the AMD may include a delivery tube having interior wall surfaces defining one or more gas channels. The gas channels may be in gas-communication with the laryngeal mask.

### PRIORITY CLAIM

This application is a continuation-in-part of and claims the benefit of priority to U.S. patent application Ser. No. 17/166,950 (filed 3 Feb. 2021), which is a continuation-in-part of Ser. No. 16/864,248 (filed 1 May 2020), which is a continuation-in-part of PCT/US2019/045830 (filed 9 Aug. 2019). The disclosures of the foregoing three patent applications are hereby incorporated by reference.

### FIELD OF THE INVENTION

The present invention relates to laryngeal mask airway devices and associated methods of maintaining an open airway in a patient.

### BACKGROUND OF THE INVENTION

In the prior art there are laryngeal mask airway devices that include a mask-portion that extends over the laryngeal inlet of a patient. Such laryngeal-mask airway devices provide an airway (i.e. a passage or pathway) through which the patient may receive breathing gas, and through which the patient may exhale gas. However, none of the prior art devices disclose or suggest features of the present invention, each of which, alone or in combination, improve the ability of the mask to facilitate use of the mask, and thereby better accommodate the patient.

For example, U.S. Publication No. US 2010/0319704 (“Nasir704”) discloses an airway device having an airway tube 11 and “a laryngeal cuff or cup 14”. The cuff/cup 14 is adapted to fit over the laryngeal inlet of a patient. Nasir704 discloses that the cuff/cup 14 has outbulgings 15, 16 positioned to fit into the piriform fossae and aryepiglottic folds and space postero-inferior to the thyroid and cricoid cartilages in order to provide a seal. However, Nasir704 does not disclose or suggest a pocket or a ridge that subtends part of the outbulgings 15, 16 and that is positioned to interact with the patient's epiglottis so that in use the patient's epiglottis is held away from the laryngeal inlet. Also, Nasir704 does not disclose or suggest a pocket or a ridge that is circumscribed by the outbulgings 15, 16. Nor does Nasir704 disclose or suggest a pocket or a ridge that extends from an area that is circumscribed by the outbulgings 15, 16. Nor does Nasir704 disclose or suggest a plurality of ridges on a posterior-side of the mask that contact the patient on the posterior hypopharynx, and thereby press the outbulgings 15, 16 against the piriform fossae and aryepiglottic folds. And, Nasir704 does not disclose or suggest a lateral compression pocket adjacent to the posterior-side of the mask for enabling the width of the cuff/cup 14 to become smaller when needed.

Another example of the prior art is U.S. Publication No. US 2004/0020491 (“Fortuna”), which discloses a laryngeal mask 12 having a supraglottic cuff 14 that provides a surface referred to by Fortuna as a peripheral seal 15. Fortuna also discloses an epiglottic band 50 that is attached to two side walls 44 of the cuff 14. The band 50 “is positioned and fixed on the side walls 44 to catch up with the epiglottis 82 and while the cuff 14 is inflating the band will push the epiglottis forward to its naturally open or flexed position as shown in FIG. 11.” See Fortuna's paragraph [0068]. Fortuna's FIG. 11 clearly shows the epiglottis 82 being held above the band 50. Consequently, Fortuna does not disclose or suggest an epiglottis-holding pocket in which at least a portion of the patient's epiglottis resides when the device is in use.

In addition, Fortuna does not disclose or suggest a ridge that (a) subtends a portion of an anterior patient-contacting surface, and (b) is positioned so that during use the ridge holds the epiglottis away from the patient's laryngeal inlet. A “ridge” is an elongated elevated part. Although Fortuna's band 50 may be said to be elongated, Fortuna's FIGS. 1, 2, 3 and 11 each shows the band 50 is not elevated. Instead, Fortuna's band 50 dips into the recess 40. As such, Fortuna does not disclose or suggest an epiglottis-holding ridge that is positioned relative to an anterior patient-contacting surface so that, when the laryngeal mask is installed in a patient, at least a portion of the epiglottis-holding ridge touches and bends the patient's epiglottis so that the epiglottis is retracted away from the patient's laryngeal inlet.

Nor does Fortuna disclose or suggest a plurality of ridges on a posterior-side of the mask that contact the patient on the posterior hypopharynx, and thereby presses the seal 15 against the tissues surrounding the laryngeal inlet. And, Fortuna does not disclose or suggest a lateral compression pocket adjacent to the posterior-side of the mask for enabling the width of the supraglottic cuff 14 to become smaller when needed.

U.S. Publication No. US 2011/0277772 (“Nasir772”) is another example of a prior art airway device. Nasir772 discloses an airway device having a laryngeal cuff 14 with a face that is shaped to fit over the patient's laryngeal inlet Nasir772 states the cuff 14 is adapted to correspond with the larynx inlet region (see Nasir772 at paragraph [0114]). Specifically, Nasir772 states the laryngeal cuff 14 is “the element of the device at the distal end of the airway tube that is adapted to cover and form a seal with the laryngeal inlet of the patient when the device is in use,” (emphasis added).

Additionally, and separate from features forming a seal is Nasir772's lip 18. According to paragraph [0125], the lip 18 “extends upwards and backwards towards the proximal end of the device, and away from the open face of the cuff. This lip 18 is sized and shaped so as to be anatomically positioned against the epiglottis and to hold the epiglottis back from folding towards the laryngeal inlet thus avoiding obstruction to airflow,” (see Nasir772 at paragraph [0125]) Nasir772 describes lip 18 as taking the form of a “leaf-like structure extending out of the laryngeal cuff and directed back toward the proximal end of the airway tube,” (emphasis added) (see paragraph [0125]). Though lip 18 contacts the patient, the lip 18 is separate from the features that form a seal with the patient's laryngeal inlet. And, most importantly, the lip 18 does not subtend those features of Nasir772 that form a seal with the patient. In addition, the lip 18 of Nasir772 is not circumscribed by those features of Nasir772 that form a seal with the patient, nor does the lip 18 of Nasir 772 extend from an area that is circumscribed by those features of Nasir772 that form a seal with the patient.

Further, Nasir772 does not disclose or suggest a plurality of ridges on a posterior-side of the mask that contact the patient on the posterior hypopharynx, and thereby presses the seal surface against the area surrounding the laryngeal inlet. And, Nasir772 does not disclose or suggest a lateral compression pocket adjacent to the posterior-side of the mask.

### SUMMARY OF THE INVENTION

The invention may be embodied as an airway management device (“AMD”). The AMD has a laryngeal-mask and a delivery tube. The laryngeal-mask has a cuff-portion, at least a portion of which is a patient contacting surface that is configured to fit snugly against a patient immediately adjacent to the patient's laryngeal inlet. When properly positioned in a patient, the patient contacting surface is nearer to the patient's front and faces the patient's front, and is therefore sometimes referred to herein as the “anterior patient contacting surface”, or “APCS” for short. The laryngeal-mask has a bowl, and in use the bowl extends over the patient's laryngeal inlet, thereby providing a space through which gas is permitted to travel to and from the patient's laryngeal inlet. In some embodiments of the invention, the laryngeal-mask is not inflatable. The delivery tube provides a pathway for gas to move to and from the bowl of the laryngeal-mask, and therefore to and from the patient's laryngeal inlet.

When the laryngeal-mask is installed in the patient, the APCS contacts the patient in an area adjacent to the patient's laryngeal inlet, and at least partially surrounds the patient's laryngeal inlet. In some embodiments of the invention, the cuff (including the APCS) may have a Shore hardness of at least 5 and not more than 50 on the A-scale.

The laryngeal mask may have one or more of the following:
(a) an esophageal sealing tab;
(b) an epiglottis-holding ridge (“EHRidge”) having a crest;
(c) an epiglottis-holding pocket;
(d) one or more side compression pockets, and/or
(e) a plurality of ridges on a posterior-side of the bowl.

The delivery tube may have one or more of the following features:
(a) one or more interior wall surfaces defining one or more gas channels;
(b) lengthwise curvature; and/or
(c) an end that is distal from the laryngeal mask, and suitable for connecting to a supply hub.

The laryngeal-mask may have an esophageal sealing tab (“ESTab”) that subtends a portion of the cuff, and/or the APCS. If a feature is described herein as subtending the cuff, it will be understood the feature may subtend the APCS. And, when a feature is described herein as subtending the APCS, the feature necessarily subtends the anterior cuff too. Finally, a feature that subtends the anterior cuff or the APCS may be circumscribed by the anterior cuff or the APCS, as the case may be.

The location of the ESTab may be distal from the delivery tube, relative to other portions of the anterior cuff. Such an ESTab may be curved. More than one such ESTab may be included. The one or more ESTabs may provide a resistance to the flow of gas, or may provide a seal with the patient, thereby encouraging gas to travel to/from the patient's laryngeal inlet.

The laryngeal-mask may have an epiglottis-holding ridge (some might prefer to call this an epiglottis retention tab) that subtends a portion of the anterior cuff, or the APCS. The epiglottis-holding ridge (“EHRidge”), and that may be proximal to the delivery tube, relative to other portions of the anterior cuff. The EHRidge may be circumscribed by the APCS, the anterior cuff, or both, or extends from an area that is circumscribed by the APCS, the anterior cuff, or both. In use, the laryngeal-mask is inserted in the patient until the crest of the EHRidge slides beyond the epiglottis, and then the laryngeal-mask is pulled back slightly so that the ridge pulls (some might say pushes) the epiglottis away from the laryngeal inlet. Such an EHRidge may be curved. More than one such EHRidge may be included.

The EHRidge may be positioned relative to the anterior cuff such that a pocket exists between the EHRidge and the subtended portion of the anterior cuff, or the APCS, as the case may be. In such embodiments, the epiglottis-holding pocket (some might prefer to call this an epiglottis retention pocket) extends from the EHRidge toward the subtended portion of the cuff, or the APCS, as the case may be. In use, the epiglottis-holding pocket (the “EHPocket”) provides a space in which some or all of the epiglottis may reside, and in so doing facilitates the ridge pulling the epiglottis away from the laryngeal inlet. In this manner, the EHRidge (and the crest in particular) may catch the patient's epiglottis, and push the patient's epiglottis to a desired position in order to facilitate delivery of gas toward the patient's lungs via the laryngeal-mask. In use, the EHRidge may push and/or hold the epiglottis away from the patient's laryngeal inlet. And the EHRidge may facilitate the patient's epiglottis residing in the EHPocket.

The EHRidge may be positioned near to the distal end of the delivery tube. For example, the positioning of the delivery tube (or distal end of a connector extending between the delivery tube to the laryngeal mask) and the EHRidge may be such that a plane that is substantially perpendicular to the primary flow direction of gas, when in use, immediately prior to leaving the delivery tube (or connector, as the case may be), intersects (a) with the distal end of the delivery tube (or connector, as the case may be), and (b) the EHRidge and/or the EHPocket. For ease of description, when used with respect to the delivery tube, the “distal end” of the delivery tube will be assumed to include the distal end of a connector or extension that is joined to the distal end of the delivery tube. And, it should be noted the invention may be accomplished by a device wherein the delivery tube and mask are integrally formed as a single piece during the manufacturing process.

The EHPocket may be positioned near the distal end of the delivery tube. For example, the distal end of the delivery tube and the EHPocket may be positioned such that a plane that is substantially perpendicular to the primary flow direction of gas, when in use, immediately prior to leaving the delivery tube intersects with (a) the EHPocket, and (b) the distal end of the delivery tube.

On a posterior-side of the mask, which may face away and/or may be on a side opposite from the APCS, there may be a plurality of ridges for facilitating placement of the mask in the patient, and/or in order to assist with pressing the APCS to the patient's tissue adjacent to the laryngeal inlet. Such plurality of posterior-side ridges may be substantially parallel to each other. When the posterior-side ridges are substantially parallel, the plurality of substantially parallel ridges may be oriented in a direction that is substantially parallel to a direction in which the delivery tube extends, and therefore substantially parallel to a direction in which the mask moves when being inserted into a patient, or substantially parallel to a direction in which the mask moves when being removed from a patient.

The plurality of posterior-side ridges may be flexible, and in that situation, some might prefer to call these ridges compression ribs. The flexibility of the posterior-side ridges may be selected so as to allow the ridges to adapt to anatomical differences between patients, and thereby better accommodate a large number of patients using masks manufactured according to a small number of configurations. These posterior-side ridges may be flexible under the forces expected to be exerted by the patient's pharynx when the mask resides in the patient. In some embodiments of the invention, the posterior-side ridges may have an aspect ratio (height: width) in a range from 0.53:1 to 22.38:1 including every increment (e.g. every 0.01 increment) therebetween of the height and/or width of the ratio. For example, in some embodiments of the invention, the ridges may have an aspect ratio of from 1.07:1 to 11.19:1, including every increment (e.g. every 0.01 increment) of the height and/or width therebetween.

In some embodiments of the invention, some of the posterior-side ridges may have a height that is nearly zero. However, in some embodiments of the invention, some of the posterior-side ridges may have a height that is 0.291 inches. The height of the posterior-side ridges of some embodiments of the invention need not be uniform, and it is expected that ridges in the center of the mask will likely have a maximum height that is greater than the heights of those posterior-side ridges positioned near lateral sides (i.e. away from the center) of the mask. Furthermore, the height of a particular posterior-side ridge need not be uniform, but instead may vary along the length of the posterior-side ridge, and this may be particularly true when it is desired to avoid abrupt transitions on the patient-facing surfaces of the posterior-side ridges. For example, the height of a posterior-side ridge may be greater in the middle of the rib than the height of that rib at either of the ends of that rib.

In some embodiments of the invention, some of the posterior-side ridges may have a width that is quite narrow (e.g. 0.034 inches). In some embodiments of the invention, some of the posterior-side ridges may have a width that is relatively wide (e.g. 0.268 inches). For a particular posterior-side ridge, it is expected that the width will not be uniform, and it is likely that the width of a posterior-side ridge will be larger near the base of that ridge where the ridge meets the posterior side of the mask than is the width of that ridge near its crest.

The delivery tube may have interior wall surfaces defining one or more gas channels (the “DT Gas Channel” or “DT Gas Channels”). The DT Gas Channel(s) is (are) in gas-communication with the space created by the bowl of the laryngeal-mask. The DT Gas Channels may be configured to facilitate conveying gas:
toward the laryngeal mask; and/or
away from the laryngeal mask.

The space created by the bowl facilitates conveying that gas to and from the patient's laryngeal inlet.

The delivery tube may be curved lengthwise. In such an embodiment of the invention, the anterior side of the laryngeal mask, which when positioned in a patient faces generally toward the patient's laryngeal inlet and the tissue immediately adjacent to that laryngeal inlet, may be on the same side as a radius of curvature that defines the lengthwise curve. See FIG. 2D.

Some embodiments of the invention, including those described above, may include a supply hub having an interior wall surface defining a gas channel (the “Hub Gas Channel”) The Hub Gas Channel may be configured to facilitate gas-communication with a source of breathing gas. The Hub Gas Channel may be configured to facilitate conveying:
(i) breathing gas toward the DT Gas Channel; and/or
(ii) exhaled gas away from the DT Gas Channel.

A supply hub that is in keeping with the invention may have interior wall surfaces defining at least two Hub Gas Channels. Each of the Hub Gas Channels may be:
(a) configured to facilitate gas-communication with a source of breathing gas; and
(b) configured to facilitate gas-communication with the delivery tube.
   For example, each of the Hub Gas Channels may be disposed and/or may be configured to facilitate conveying breathing gas toward the delivery tube and/or exhaled gas away from the delivery tube. In particular, in an embodiment of the invention having two Hub Gas Channels, each Hub Gas Channel may be configured to facilitate conveying:
(a) breathing gas toward the DT Gas Channel(s); and
(b) exhaled gas away from the DT Gas Channel(s).

In some embodiments of the invention, the supply hub may further comprise a flange. Such a flange may be used by a health care professional to apply force needed to guide and install the AMD in a patient, and/or guide and remove the AMD from the patient.

In some embodiments of the invention, including those identified above, the AMD may have a connector extending between the laryngeal-mask and the delivery tube. The connector may include interior wall surface(s) defining one or more gas channels (the each, a “Connector Gas Channel”). Each of the Connector Gas Channels may be:
configured to facilitate gas-communication with the delivery tube; and
configured to facilitate gas-communication with the laryngeal mask.

### Further, the Connector Gas Channels May be Configured to Facilitate Conveying Gas:

from the delivery tube toward the laryngeal mask; and
from the laryngeal mask toward the delivery tube.

The delivery tube of such an AMD may have interior wall surfaces defining at least two gas channels (each, a “DT Gas Channel”). Each DT Gas Channel may be configured to facilitate gas-communication with the Connector Gas Channels. For example, the DT Gas Channels may be configured to facilitate conveying gas toward and away from the Connector Gas Channels. And, even more specifically:
(a) a first DT Gas Channel may be configured to facilitate gas-communication with a first Connector Gas Channel, but not the second Connector Gas Channel; and
(b) a second DT Gas Channel may be configured to facilitate gas-communication with a second Connector Gas Channel, but not the first Connector Gas Channel.

With regard to the connector of such an AMD:
(a) a portion of the connector may reside inside the delivery tube, or a portion of the delivery tube may reside inside the connector; and
(b) a portion of the connector may reside inside the laryngeal mask, or a portion of the laryngeal mask may reside inside the connector.

For those embodiments of the invention that are not inflatable, the anterior cuff of the laryngeal-mask may have a Shore hardness of at least 5 and not more than 50 on the A-scale. In particular, when positioned in a patient, that portion of the laryngeal mask that touches the tissue immediately adjacent to that patient's laryngeal inlet, in order to facilitate forming a seal with that tissue, may have a Shore hardness of at least 5 and not more than 50 on the A-Scale.

### BRIEF DESCRIPTION OF THE DRAWINGS

For a fuller understanding of the nature and objects of the invention, reference should be made to the accompanying drawings and the subsequent description. Briefly, the drawings are:

FIG. 1 is a schematic of an airway management device that is in keeping with the invention;

FIG. 2A is a perspective view of an airway management device that is in keeping with the invention;

FIG. 2B is a front view of the airway management device that is shown in FIG. 2A;

FIG. 2C is a top view of the airway management device that is shown in FIG. 2A;

FIG. 2D is a cross-sectional view taken along the line 2D-2D in FIG. 2C;

FIG. 3A is a top view of a delivery tube that is in keeping with the invention;

FIG. 3B is a cross-section of the delivery tube taken along the line 3B-3B in FIG. 3A;

FIG. 3C is a cross-section of the delivery tube taken along the line 3C-3C in FIG. 3B;

FIG. 3D is an end view of another delivery tube that is in keeping with the invention;

FIG. 4A is a perspective view of a supply hub that is in keeping with the invention;

FIG. 4B is another perspective view of the supply hub depicted in FIG. 4A;

FIG. 4C is an end view of the supply hub of FIG. 4A viewed from the proximal end of the supply hub;

FIG. 4D is a top view of the supply hub of FIG. 4A;

FIG. 4E is an end view of the supply hub of FIG. 4A viewed from the distal end of the supply hub;

FIG. 4F is a cross-sectional view of the supply hub taken along the line 4F-4F of FIG. 4E;

FIG. 4G is a cross-sectional view of the supply hub taken along the line G-G of FIG. 4E;

FIG. 5A is a perspective view of the supply hub assembly of FIG. 5A showing a portion of the delivery tube mated with the supply hub; Figure SB is a top view of the assembly depicted in FIG. 5A;

FIG. 5C is an end view of the assembly depicted in FIG. 5A viewed from the proximal end of the supply hub;

FIG. 5D is an end view of the assembly depicted in Figure SA viewed from the distal end of the supply hub;

FIG. 5E is a cross-section of the supply hub taken along the line 5E-5E in FIG. 5D;

FIG. 6A is a perspective view of a laryngeal mask according to the invention;

FIG. 6B is a perspective view of the mask depicted in FIG. 6A viewed from a distal end of the mask;

FIG. 6C is an end view of the mask depicted in FIG. 6A viewed from a distal end of the mask;

FIG. 6D is an end view of the mask depicted in FIG. 6A viewed from a proximal end of the mask;

FIG. 6E is a bottom view of the mask depicted in FIG. 6A showing a posterior-side of the mask;

FIG. 6F is a side view of the mask depicted in FIG. 6A;

FIG. 6G is a top view of the mask depicted in FIG. 6A showing an anterior-side of the mask;

FIG. 6H is a cross-section of the mask taken along the line 6H-6H in FIG. 6G;

FIG. 7A is a perspective view of another mask that is in keeping with the invention;

FIG. 7B is a different perspective view of the mask depicted in FIG. 7A showing the posterior-side of the mask;

FIG. 7C is a top view of the mask depicted in FIG. 7A showing the anterior side of the mask;

FIG. 7D is a bottom view of the mask depicted in FIG. 7A showing the posterior side of the mask;

FIG. 7E is an end view of the mask depicted in FIG. 7A viewed from the distal end of the mask;

FIG. 7F is a side view of the mask depicted in FIG. 7A;

FIG. 7G is a cross-section taken along the line 7G-7G in FIG. 7F;

FIG. 8A is a perspective view of an AMD that is in keeping with the invention, and which has a connector interfacing between the mask and the delivery tube;

FIG. 8B is a is another perspective view of the AMD of FIG. 8A;

FIG. 8C is a top view of the AMD of FIG. 8A showing the anterior side of the mask;

FIG. 8D is a cross-section taken along the line 8D-8D in FIG. 8C;

FIG. 8E is an end view of the AMD of FIG. 8A viewed from the distal end of the mask;

FIG. 8F is a perspective view that depicts a proximal side of the connector shown in FIGS. 8A-8E that may be used between the mask and the delivery tube;

FIG. 8G is a perspective view of the connector depicted in FIG. 8F viewed from a distal side of the connector;

FIG. 8H is an end view of the connector depicted in FIG. 8F showing a proximal end of the connector;

FIG. 8I is an end view of the connector depicted in FIG. 8F showing a distal end of the connector;

FIG. 8J is a cross-section of the connector taken along the line 8I-8J in FIG. 8I;

FIG. 8K is a cross-section of the connector taken along the line 8K-8K in FIG. 8I;

FIG. 9 is a flow diagram depicting a method that is in keeping with the invention;

FIG. 10 depicts a patient and certain features of the patient's respiratory system;

FIG. 11 depicts an AMD according to the invention while a laryngeal mask is inserted into the patient's mouth (an arrow near the patient's oropharynx shows the direction in which the mask is being pushed);

FIG. 12 depicts the AMD of FIG. 11 at a later point in time when the laryngeal mask is being pushed through the patient's oropharynx into the patient's hypopharynx (an arrow near the patient's oropharynx shows the direction in which the mask is being pushed);

FIG. 13A depicts the AMD of FIGS. 11 and 12 at a later point in time when a distal tip of the laryngeal mask is being pushed into the patient's esophagus (the large arrow shows the direction in which the mask is being pushed);

FIG. 13B depicts the AMD of FIGS. 11, 12, and 13A at a later point in time when the laryngeal mask is being pulled back slightly so as to catch a tip of the patient's epiglottis in a pocket of the laryngeal mask (the large arrow shows the direction in which the mask is being pulled); and

FIG. 13C depicts the AMD of FIGS. 11, 13A, and 13B at a later point in time when the laryngeal mask is no longer being pushed or pulled, and has been positioned so that a bowl of the laryngeal mask coincides with and faces the patient's glottis, and air is in the AMD traveling to and from the patient's lungs, or more specifically breathing gas supplied to the AMD moves toward the patient's lungs and exhaled gas from the patient's lungs moves away from the patient's lungs.

### Further Description of the Invention

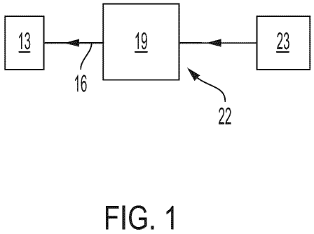

The invention may be embodied as an airway management device (“AMD”) 10 for maintaining access to a patient's laryngeal inlet. For example, the invention may be embodied as an AMD 10 for maintaining access to the laryngeal inlet of an infant. Such a device 10 may be a laryngeal mask 13, or a laryngeal mask 13 in combination with a delivery tube 16, or may be a laryngeal mask 13 in combination with both a delivery tube 16 and a supply hub 19. FIG. 1 schematically depicts all three: a laryngeal mask 13, a delivery tube 16, and a supply hub 19. The supply hub 19 may provide a gas port 22 for connecting to a source 23 of breathing gas.

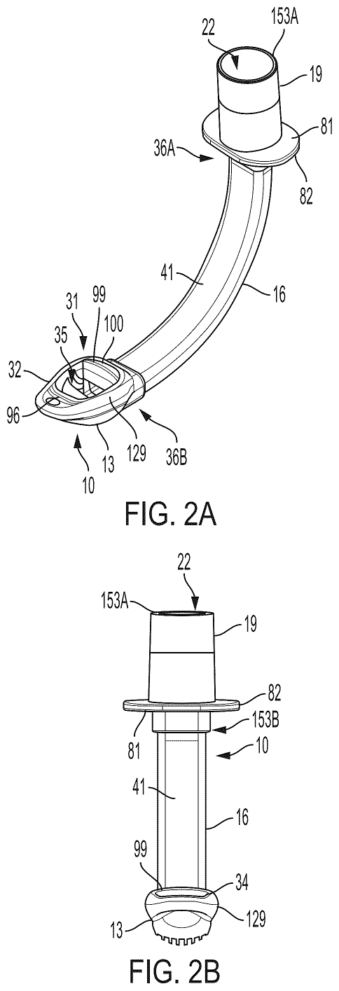

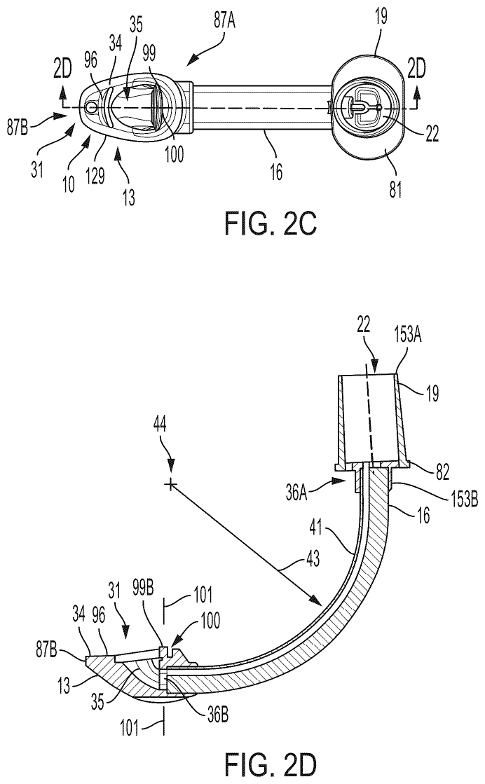

FIGS. 2A-2D depict an embodiment of the invention in the form of a device 10 for maintaining access to a patient's laryngeal inlet. The embodiment of FIGS. 2A-2D has a laryngeal mask 13, a delivery tube 16, and a supply hub 19. The laryngeal mask 13 may be shaped to fit snugly against the area proximal to and surrounding the patient's (such as a human being's) laryngeal inlet. This particular device 10 may be sized and shaped to accommodate an infant of 5 kilograms or less. The device 10 may be provided in four (or more) sizes. One size may be for infants having a very small pharynx and laryngeal inlet (typically such an infant weighs less than 1000 grams). A second size may be for infants having a small pharynx and laryngeal inlet (typically such an infant weighs from 1000 to 2000 grams). A third size may be for infants having a medium sized pharynx and laryngeal inlet (typically such an infant weighs from 2000 to 3500 grams). A fourth size may be for infants having a larger pharynx and laryngeal inlet (typically such an infant weighs from 3500 grams to 5000 grams).

The delivery tube 16 may be mated with or integrally formed with the laryngeal mask 13 and/or the supply hub 19. If not integrally formed with the mask 13 and/or the supply hub 19, the delivery tube 16 may be secured to one or both of the mask 13 and/or supply hub 19 by an adhesive such as an adhesive that is curable with ultra-violet light, for example, Loctite AA 3971. The delivery tube 16 may be made of Polyvinyl Chloride (often referred to as “PVC”), such as Teknor Apex 3301R-81-NT. The supply hub 19 may be made of Polycarbonate, such as Makrolon 2485 clear.

In some embodiments of the invention, the mask 13 may not be inflatable. A face-region 31 of the mask 13 provides a cuff 32, which in use provides an anterior patient-contacting surface 34 that contacts the patient near the patient's laryngeal inlet, may be made of a deformable material (e.g., a Thermoplastic Elastomer (“TPE”), such as Teknor Apex Medalist MD-1213OH Natural) so as to facilitate the formation of a seal between the patient and the mask 13, at least in those locations where the APCS 34 contacts the tissue near the patient's laryngeal inlet. Such a material used to provide such an APCS 34 may have a Shore hardness on the A-scale of at least 5 and not more than 50. The mask 13 is shaped to form a bowl 35 that extends from a distal end 36B of the delivery tube 16 toward the APCS 34.

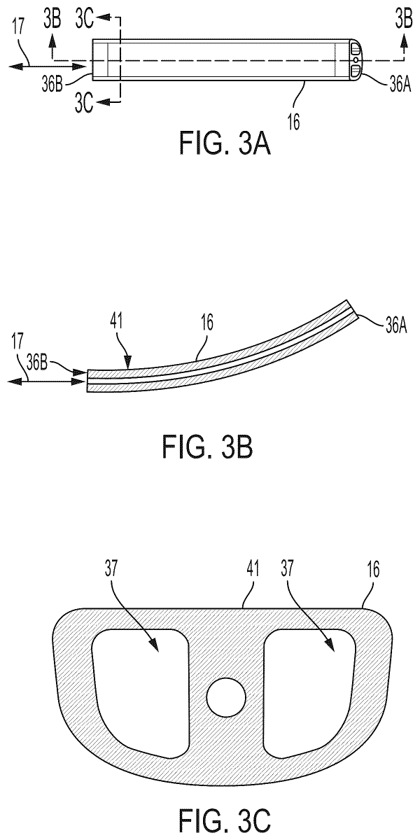

With reference to FIGS. 3A-3D, the delivery tube 16 may have one or more channels 37 that may be in gas-communication with the mask 13. Such gas channels 37 of the delivery tube 16 may be configured to facilitate conveying gas toward the mask 13 and away from the mask 13. For example, gas channels 37 of the delivery tube 16 may be used to deliver breathing gas toward the mask 13 as the patient inhales, and used to deliver exhaled gas away from the mask 13 as the patient exhales.

The delivery tube 16 may be shaped to have a substantially flat exterior side 41, which is substantially flat when viewed in a cross-section taken substantially perpendicular to an in-use primary flow direction 17 of the gas within the delivery tube. Such a substantially flat side 41 may facilitate gripping of the delivery tube 16 by a healthcare professional, and may be used by the healthcare professional to facilitate proper orientation of the device 10 prior to inserting and during insertion of the mask 13 into a patient.

The delivery tube 16 may be curved lengthwise and flexible so as to substantially conform to the general curvature of the patient's pharynx. The delivery tube 16 may be manufactured to have a pre-existing curvature (i.e a curve that exists without applying any external force to the AMD 10), and that curvature may subtend an angle of from 25 degrees to 135 degrees. The radius of curvature 43 of the delivery tube 16 may be from 1 18 inches to 1.59 inches, including all increments (e.g. every 0.01 inch increment) therebetween. The AMD 10 may be manufactured (which may include assembling) so that the mask 13 is oriented relative to the delivery tube 16 so that the face-region 31 is oriented generally toward a center of curvature 44 corresponding to the delivery tube's 16 radius of curvature 43: FIG. 2D shows this aspect. In some embodiments of the invention, the delivery tube 16 may have at least two gas channels 37.

In some embodiments of the invention, the radius of curvature 43 of the delivery tube 16 is, without applying any external force to the AMD 10, smaller than an average radius of curvature generally corresponding to the patient's pharynx. By having such a radius of curvature 43, the patient's pharynx will press on the delivery tube 16 so as to increase the delivery tube's 16 radius of curvature 43. When not in its natural radius of curvature 43 by virtue of the pharynx pressing on the delivery tube, the tube's 16 tendency to return to its natural radius of curvature 43 may supply a force to the mask 13, and this causes the APCS 34 to press against the tissue surrounding the patient's laryngeal inlet, thereby encouraging the APCS 34 to form a seal in those locations where the APCS 34 contacts the tissue adjacent to the patient's laryngeal inlet.

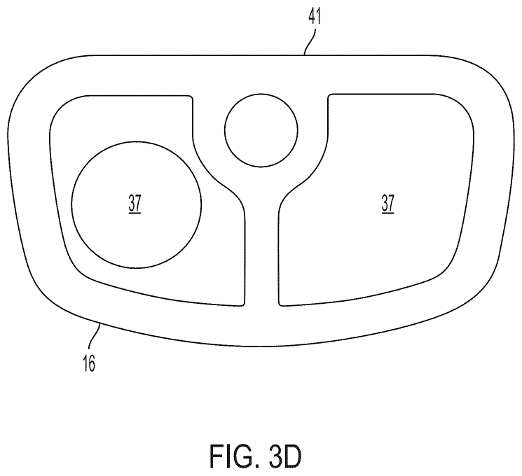

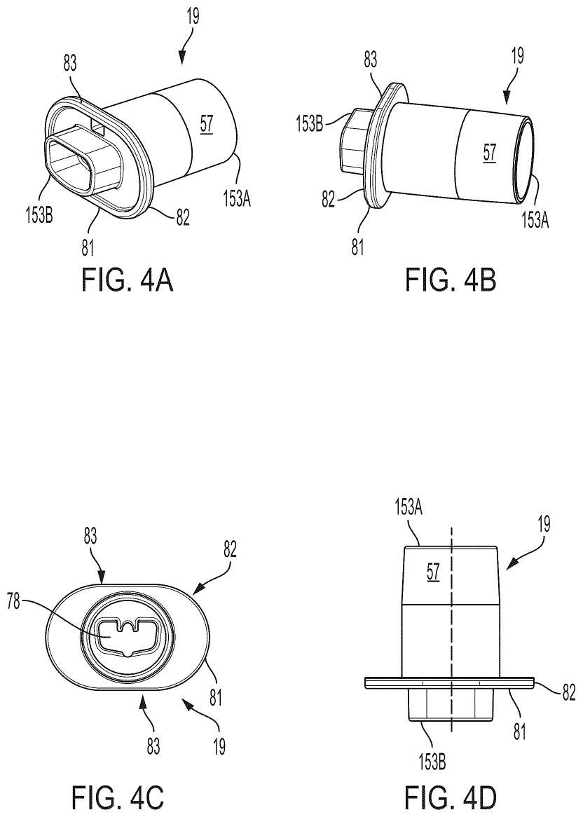

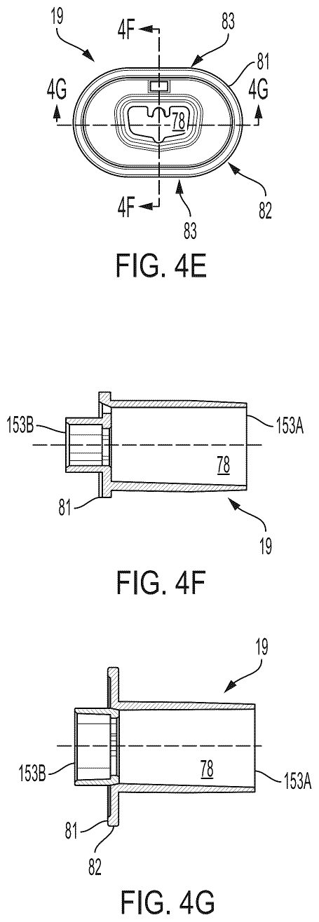

FIGS. 4A-4G depict a supply hub 19 that is in keeping with the invention. Via the supply hub gas channel 78, gas may travel to and from the delivery tube 16. Figures SA-5E depict a supply hub 19 having a single gas channel 78. However, the invention is not limited to such a design.

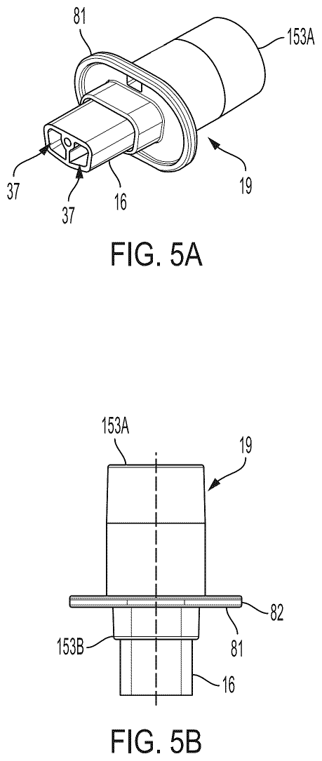

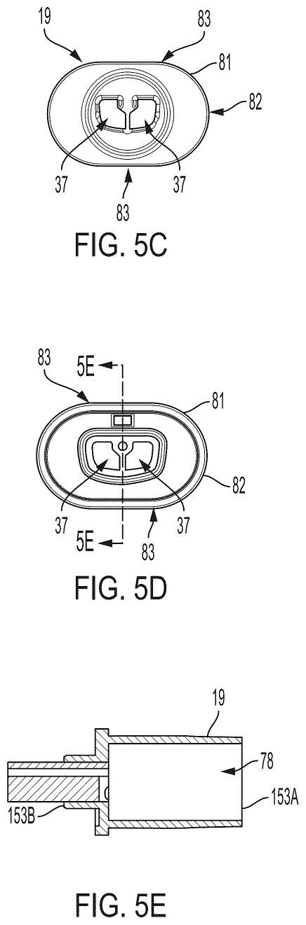

The supply hub 19 may have a flange 81 (see e.g. FIGS. 5A-5E) that extends away from a central location of the supply hub 19. When the supply hub 19 is engaged with the delivery tube 16 and the delivery tube 16 is engaged with the mask 13, the hub flange 81 may be used by a health care professional as a location against which to push and/or twist in order to insert into and orient the mask 13 relative to a patient. To facilitate such pushing and/or twisting to achieve proper orientation and positioning of the AMD 10, the flange 81 may have an annularly positioned surface 82, part of which may be substantially flat 83.

To accommodate inhalation by the patient, breathing gas can be delivered from a gas source 23 to the supply hub 19, which in turn may deliver the breathing gas via the hub gas channel 78 to the delivery tube 16, which in turn may deliver the breathing gas via the delivery tube gas channel(s) 37 to the bowl 35 of the mask 13. In addition, to accommodate exhalation by the patient, exhaled gas from the patient may be delivered to the bowl 35 of the mask 13, which in turn may deliver the exhaled gas to the delivery tube 16, which in turn may deliver the exhaled gas via the delivery tube gas channel(s) 37 to the hub gas channel 78.

FIGS. 5A through 5E depict a supply hub 19 and a portion of a delivery tube 16 mated with the supply hub 19. The supply hub 19 may be configured to serve as a place for connecting a supply of gas so that gas, such as breathing gas, can be provided to the delivery tube 16. The supply hub 19 may also be configured to deliver exhaled gas to a proper conduit. The supply hub 19 may include a flange 81, which may be used by a physician to apply pressure needed to insert the mask 13 into a patient, and later to apply pressure needed to remove the mask 13 from the patient.

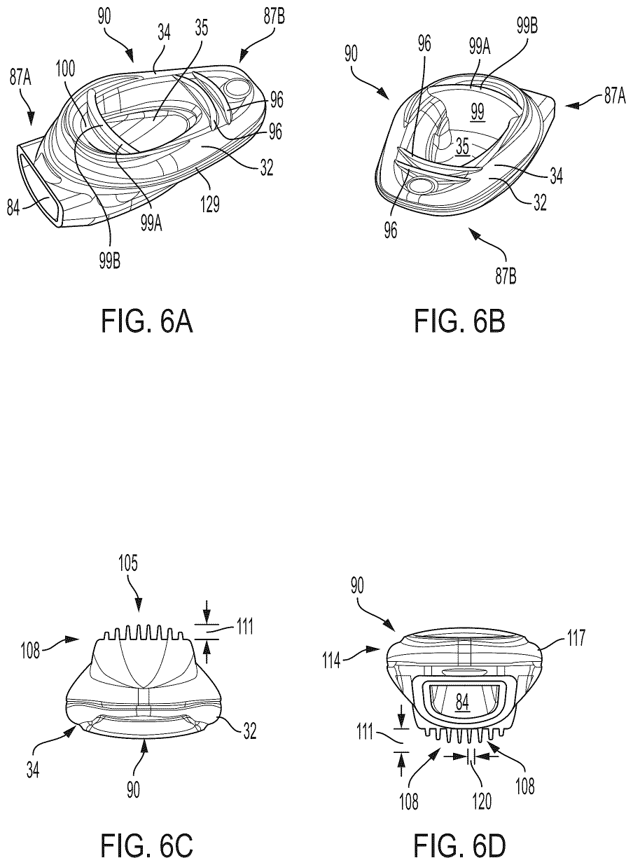

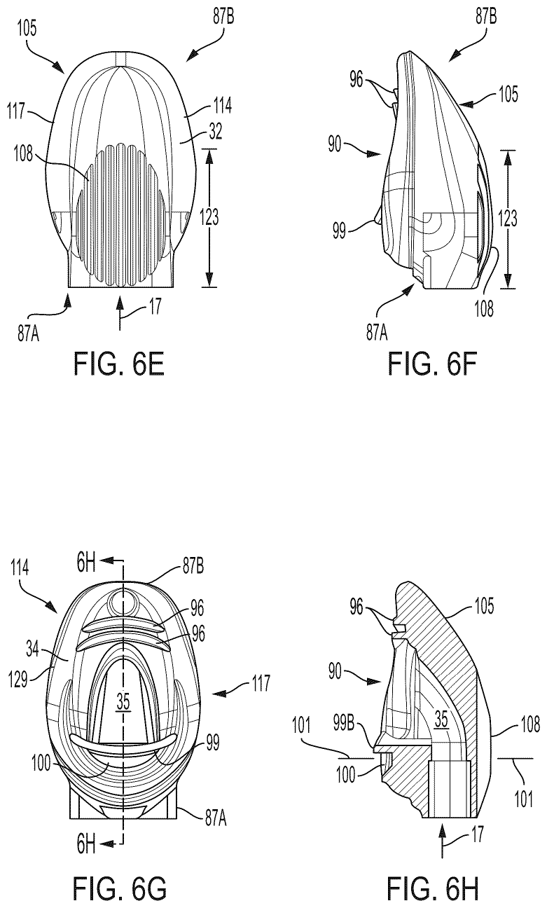

FIGS. 6A-6H depict a mask 13 that is in keeping with the invention. Such a mask 13 may have an opening 84 for receiving the delivery tube 16 (or commenter extending between the delivery tube 16 and the laryngeal mask 13) on a proximal side 87A of the mask 13 (i.e. a side of the mask 13 where the delivery tube 16 enters the mask 13), and may have an APCS 34 on an anterior-side 90 of the mask 13. The anterior patient-contacting surface 34 may be shaped to contact the patient adjacent to the patient's laryngeal inlet. When in use, the APCS 34 preferably (but not necessarily) forms a seal with the patient's tissue that is immediately adjacent to the patient's laryngeal inlet, and at least partially surrounds or preferably surrounds the patient's laryngeal inlet.

To facilitate delivery of breathing gas to the patient, the mask 13 may have one or more ridges 96, each such ridge 96 being referenced herein sometimes as an “esophageal sealing tab”, or “ESTab”. The ESTab(s) 96 may subtend a portion of the anterior cuff 32 and/or the APCS 34 that is distal from the delivery tube 16 relative to other portions of the APCS 34. See FIGS. 6A, 6B, and 6G. In FIGS. 6A, 6B, and 6G, the ESTab(s) 96 is/are shown circumscribed by both the cuff 62 and the APCS 34. The ESTab(s) 96 may facilitate a seal between the mask 13 and the patient, and may serve to direct breathing gas toward the patient's laryngeal inlet.

The location of the ESTab(s) 96 may be distal from the delivery tube 16, relative to other portions of the APCS 34, such as the EHRidge 99 and/or EHPocket 100 Such an ESTab 96 may be curved. FIGS. 2C, 6G, and 7C show one manner in which the ESTab 96 may be curved. In those figures it will be noticed that the ESTab 96 is curved so that a medial portion of the ESTab 96 is closer to the distal side 87B than are the lateral portions of the ESTab 96, which are near to the APCS 34. Another way in which the ESTab 96 may be curved results (when in use) in the medial portion of the ESTab 96 extending from the mask 13 (toward the patient's esophagus) to a greater degree than do the lateral portions of the ESTab 96 that are nearer to the APCS 34.

To facilitate holding the patient's epiglottis away from the laryngeal inlet, the mask 13 may have one or more EHRidges 99 that subtend a portion of the anterior cuff 32 and/or the APCS 34 on the proximal side 87A of the mask 13. In such an arrangement, the anterior cuff 32 circumscribes the one or more EHRidges 99. In the arrangement shown in FIGS. 6A-6H, the EHRidges 99 extend from an area that is circumscribed by the anterior cuff 32. By subtending a portion of the anterior cuff 32, or the APCS 34, an epiglottis-holding pocket (a.k.a. “EHPocket”) 100 may be formed in part by a surface of the EHRidge 99, and the EHPocket 100 may extend from that EHRidge 99 toward the subtended portion of the anterior cuff 32 or the APCS 34, as the case may be. FIGS. 6A, 6G, and 6H indicate the EHPocket 100 may be located between the EHRidge 99 and the APCS 34 on a side of the mask 13 that is adjacent to the delivery tube 16. When in use, the patient's epiglottis may reside in the EHPocket 100.

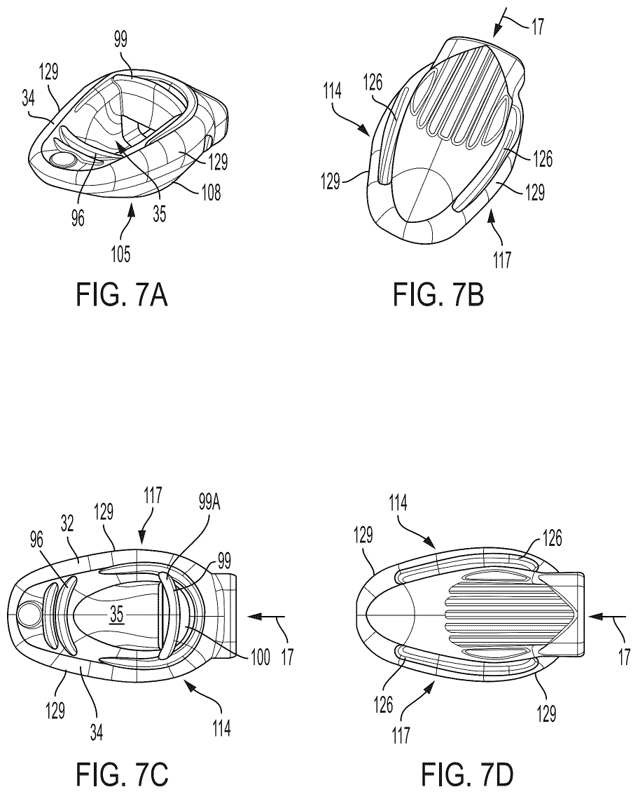

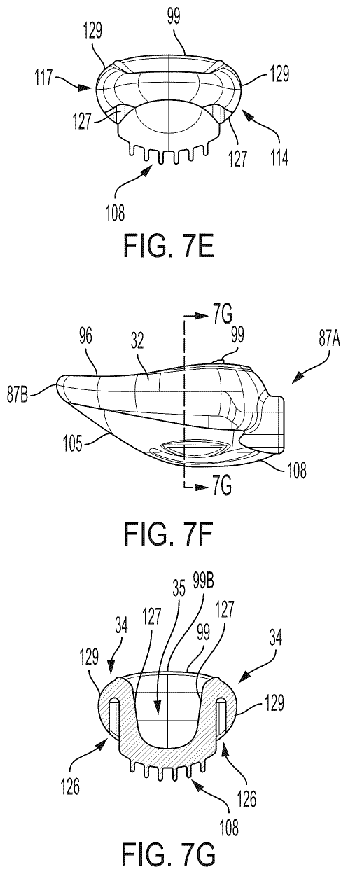

Such an EHRidge 99 may be curved. FIGS. 2C, 6G, and 7C show one manner in which the EHRidge 99 may be curved. In those figures it will be noticed that the EHRidge 99 is curved so that a medial portion of the EHRidge 99 is closer to the proximal side 87A than are the lateral portions of the EHRidge 99, which are near to the APCS 34. Another way in which the EHRidge 99 may be curved is shown in FIG. 7G. That curvature results (when in use) in the medial portion of the EHRidge 99 extending from the mask 13 (toward the patient's epiglottis) to a greater degree than do the lateral portions of the EHRidge 99 that are nearer to the APCS 34. See also FIGS. 2D, 6F, 6H, 7E, 7F, 7G, and 8D.

The EHRidge 99 may have a curved surface 99A that extends (in use) toward the patient and beyond the APCS 34 so as to crest in a location 99B that is at a medial location of the EHRidge 99 and approximately equi-distant between the lateral sides 114, 117 of the mask 13. By extending in this fashion, the EHRidge 99 may better able to catch and hold the patient's epiglottis.

The EHRidge 99 may be positioned near to the distal end 36B of the delivery tube 16 (i.e. on the proximal side 87A of the laryngeal mask 13. The distal end 36B of the delivery tube 16 and at least a portion of the EHRidge 99 may reside in a plane 101 that is substantially perpendicular to the primary flow-direction 17 of gas, when in use, flowing to and from the delivery tube 16 at a location that is immediately prior to exiting the delivery tube 16 toward the mask 13. For example, the plane 101 may be (a) substantially perpendicular to the primary flow-direction 17 of gas, when in use, immediately prior to leaving the delivery tube 16, and (b) intersect with least a portion of the distal end 36B of the delivery tube 16, and (c) intersect with the EHRidge 99 and/or the EHPocket 100.

In embodiments of the invention, the EHPocket 100 may be positioned near to the distal end 36B of the delivery tube 16, which is located on the proximal side 87A of the laryngeal mask 13. This relative positioning of the EHPocket 100 and distal end 36B may be such that a plane 101 that is substantially perpendicular to the primary flow-direction 17 of gas, will pass through or at the distal end 36B of the delivery tube 16 and at least a portion of the EHPocket 100. By positioning the distal end 36B of the delivery tube 16 in this manner, when in use in a patient, the distal end 36B of the delivery tube 16 will be close to the patient's laryngeal inlet.

On a posterior-side 105 of the mask 13 (the side opposite the face-region 31) may be a plurality of ridges 108, which may be oriented in a direction that is substantially parallel to a direction in which the delivery tube 16 extends. The ridges 108 of the posterior-side 105 may extend from a posterior-side of the bowl 35. Such posterior-side ridges 108 may be made of a soft and flexible material (such as a Thermoplastic Elastomer (often referred to as “TPE”), an example of which is Teknor Apex Medalist MD-1213OH Natural) in order to facilitate placement of the mask 13 in the patient, and/or in order to assist with pressing the APCS 34 to the tissue adjacent to the laryngeal inlet. For example, the posterior-side ridges 108 may have a Shore hardness on the A-scale of at least 5 and not more than 50. The posterior-side ridges 108 may be made from the same material as other portions of the mask 13.

The height 111 of the posterior-side ridges 108 may vary. For example, posterior-side ridges 108 near the left lateral-side-portion 114 and the right lateral-side-portion 117 of the cuff 32 may have a maximum height 111 that is less than a maximum height 111 of posterior-side ridges 108 positioned centrally (i.e. medially) on the laryngeal mask 13. FIGS. 6C and 6D illustrate such a variation in height 111. Note: the labels “left” and “right” are chosen to coincide with the patient's left and right when the mask 13 is installed.

Although the widths 120 of the posterior-side ridges 108 are shown in FIGS. 6C, 6D, 6E, 7B, 7D, and 7E having substantially the same width, the width of a particular posterior-side ridge 108 need not be the same as others of the ridges 108. Furthermore, the height and/or width of the posterior-side ridges 108 may be selected to provide a desired flexibility to the ridges 108, and/or to facilitate molding of the laryngeal mask 13. For example, an aspect ratio (height: width) of the posterior-side ridges 108 may be in a range from 0.53:1 to 22.38:1, including all increments (e.g. every 0.01 increment) of the height and/or width therebetween. It is believed that the aspect ratio is likely to range from 1.07:1 to 11.19:1, including all increments (e.g. every 0.01 increment) of the height and/or width therebetween. Further, the shape of the posterior-side ridges 108 need not be triangular, as shown in the figures; rather, the posterior-side ridges 108 may have other shapes (e.g. rectangular). The space between adjacent posterior-side ridges 108 may range at the crests of the ridges 108 from 0.016 inches to 0.106 inches, including all increments (e.g. every 0.001 inch increment) therebetween. It is believed that a preferred distance between crests of the ridges 108 is likely to range from 0 032 inches to 0.053 inches, including all increments (e.g. every 0.001 inch increment) therebetween.

Furthermore, the lengths 123 of the posterior-side ridges 108 may vary. For example, posterior-side ridges 108 nearest to the left and right lateral-side-portions 114, 117 of the cuff 32 may be shorter in length 123 than posterior-side ridges 108 positioned centrally on the mask 13. FIGS. 6E and 7D best illustrates this variation in length 123.

FIGS. 7A-7G depict a mask 13 that is in keeping with the invention. Two side-grooves 126 (some might prefer to call these lateral compression pockets) are formed in part by the left and right lateral-side-portions 114, 117 of the cuff 32, and also in part by lateral-sides of the bowl 35. See, FIGS. 7B, 7D and 7G. The side-grooves 126 may be positioned to open in a direction that is generally the direction in which the posterior-side 105 faces, and in the general direction in which the posterior-side ridges 108 extend from the posterior-side of the bowl 35. Some might say the orientation of the side-grooves 126 is such that each side-groove 126 extends from a closed side of the side-groove 126 (near the APCS 34) to an open side of the side-groove 126 (distal from the APCS) in a direction that is away from the APCS 34.

Each side-groove 126 is formed in substantial part by a respective lateral-side 127 of the bowl 35 and a respective lateral-side-portion 114, 117, each lateral-side-portion 114, 117 extending from a different portion of the APCS 34. The side-grooves 126 may be positioned so that the lateral-side-portions 114, 117 are able to more easily flex toward the bowl 35 when the mask 13 is being moved through a narrow portion of the patient. These side-grooves 126 enable the lateral-side 129 of the cuff 32 (and more specifically, the lateral-side-portions 114, 117) to flex side-to-side more easily than a cuff 32 that does not have such side-grooves 126. The ability of the lateral-side-portions 114, 117 to flex in the region where the side-grooves 126 reside may facilitate installing the mask 13 in a patient and removing the mask 13 from a patient because the lateral-side-portions 114, 117 can move toward the center of the mask 13 (i.e. toward the bowl 35), thereby allowing the mask 13 to become narrower in order to move through and accommodate narrow portions of the patient's airway while also keeping the mask 13 centered in the patient. Such an ability to flex also allows a particularly-sized mask 13 to accommodate patients of different sizes and shapes.

FIGS. 2A-2D depict an AMD 10 that is in keeping with the invention. That embodiment of the invention has a delivery tube 16 that mates directly with the mask 13. In that embodiment of the invention, a distal portion of the delivery tube 16 (including the distal end 36B) fits within a proximal portion of the mask 13. However, it should be noted that this arrangement may be altered so that a proximal portion of the mask 13 (located on a proximal side 87A) fits within a distal portion of the delivery tube 16. Or, the delivery tube 16 and mask 13 may be integrally formed as a single piece during the manufacturing process.

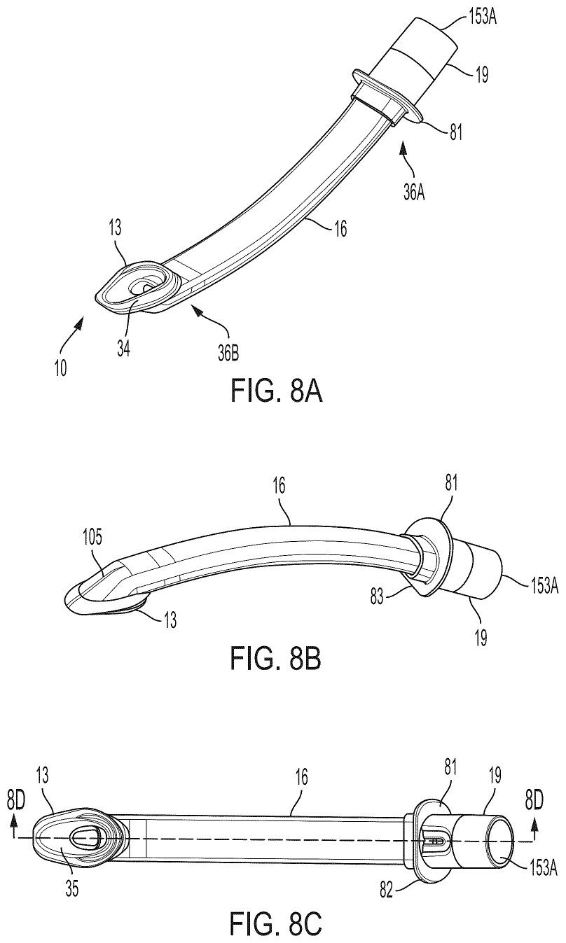

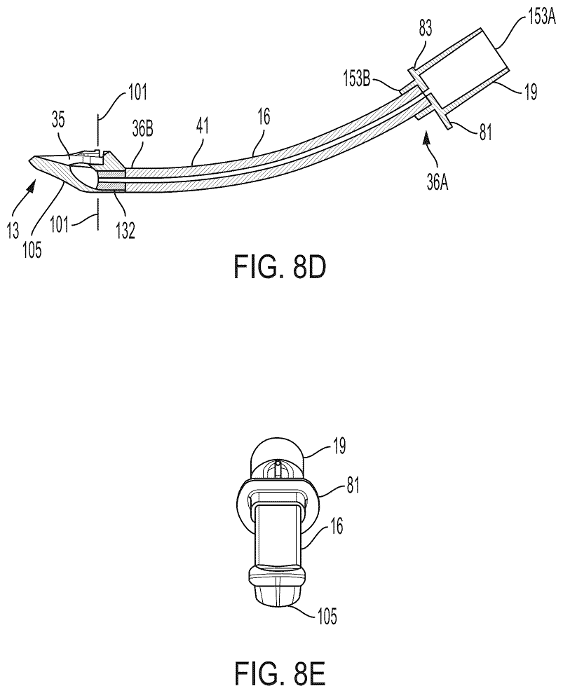

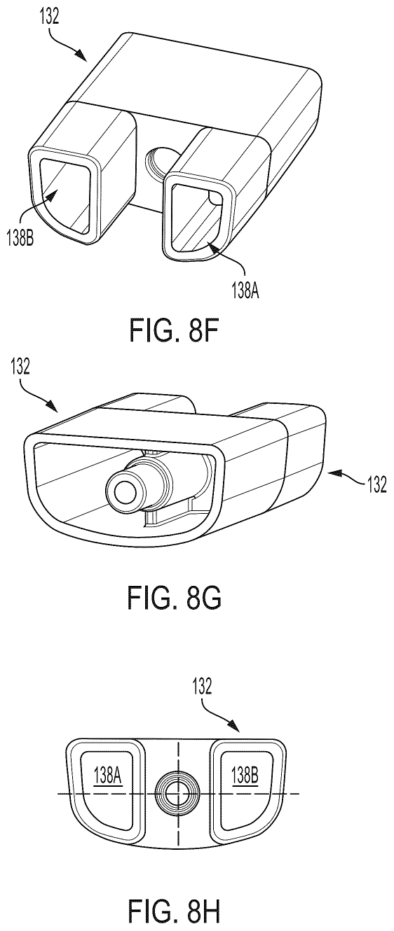

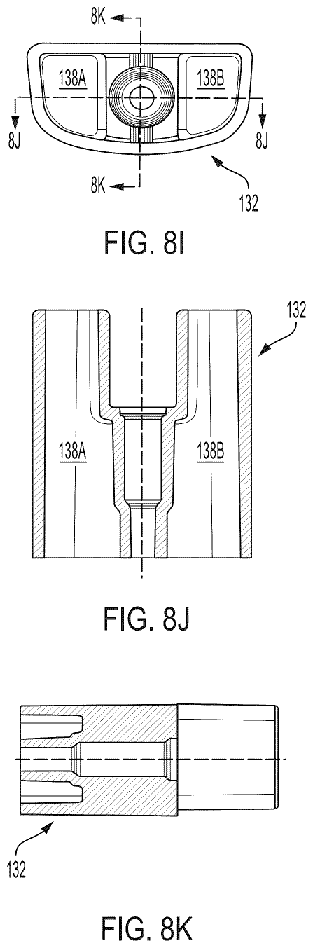

Alternatively, a connector may be used in order to facilitate a connection between the mask 13 and the delivery tube 16. FIGS. 8A-8E depict an AMD 10 having such a connector 132. In that embodiment of the invention, the connector 132 may interface with the mask 13 and delivery tube 16 so that a first portion of the connector 132 resides inside the delivery tube 16, and a second portion of the connector 132 resides inside the mask 13. However, such a connector may:
(a) interface with the mask in a way such that a proximal portion of the mask 13 resides inside a portion of the connector 132; and/or
(b) interface with the delivery tube such that the distal end of the delivery tube 16 resides inside the connector 132.
   A connector 132 situated between the mask 13 and the delivery tube 16 may have one or more gas channels 138A, 138B. Each of the gas channels 138A, 138B of the connector 132 may be configured to facilitate movement of gas from the mask 13 toward the delivery tube 16, and from the delivery tube 16 toward the mask 13. Each gas channel 138A, 138B of such a connector 132 may be configured to facilitate gas-communication with only one of the gas channels 37 of the delivery tube 16.

Additional information about the connector 132 can be obtained from FIGS. 8F-8K. Such a connector 132 also may have a first gas channel 138A and a second gas channel 138B that may be configured to facilitate gas-communication with the delivery tube 16, and gas-communication with the mask 13.

With regard to the supply hub 19, the delivery tube 16 may mate directly with the supply hub 19, and thereby facilitate the movement of gas from the supply hub to the delivery tube 16, and vice versa. Alternatively, a connector may be used to facilitate the movement of gas to and from the delivery tube 16. If a connector is used, the delivery tube 16 may be mated with a first end of the connector, and the supply hub may be mated with a second end of the connector. In this manner, the connector extends between the supply hub 19 and the delivery tube 16, and provides one or more channels through which breathing gas may be delivered to the delivery tube 16, and exhaled gas may be received from the delivery tube 16.

A connector that interfaces with the delivery tube 16 and supply hub 19 may have a portion of the connector residing inside the delivery tube 16, and a different portion of the connector residing inside the supply hub 19. However, such a connector may:
(a) interface with the supply hub 19 in a way such that a portion of the supply hub 19 resides inside a portion of the connector; and/or
(b) interface with the delivery tube 16 such that a portion of the delivery tube 16 resides inside the connector

Above it was noted the invention may be embodied as an AMD 10 having a delivery tube 16 that has a single gas channel 37. In such an AMD 10, the mask may have either the ESTab 96 or the EHRidge 99, or both of those ridges 96, 99. Others of the features described above may be included in such an AMD 10.

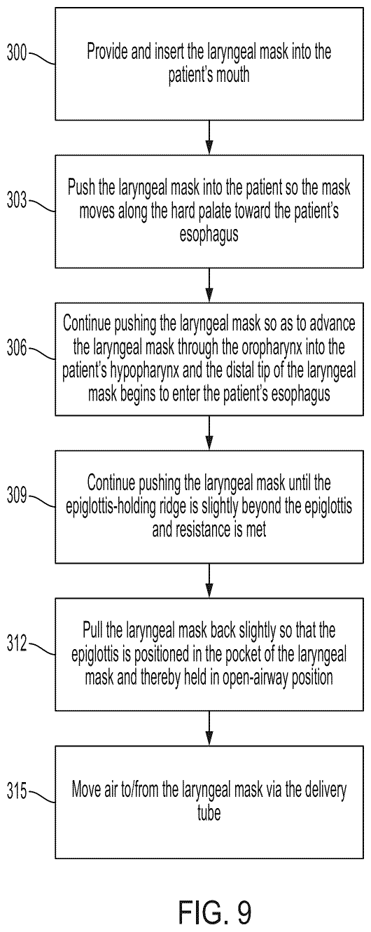

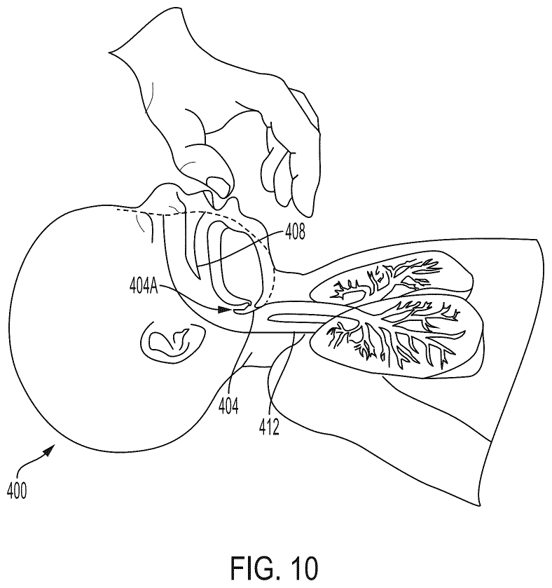

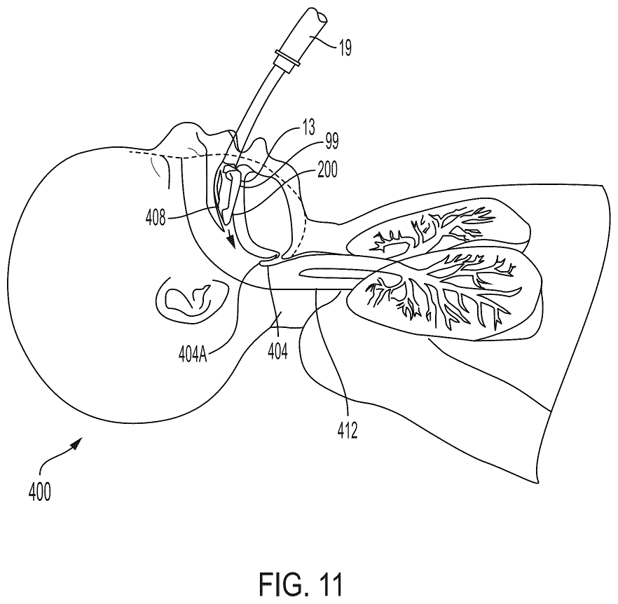

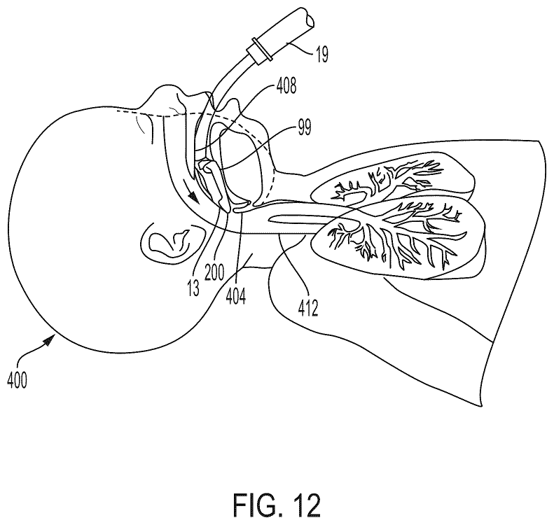

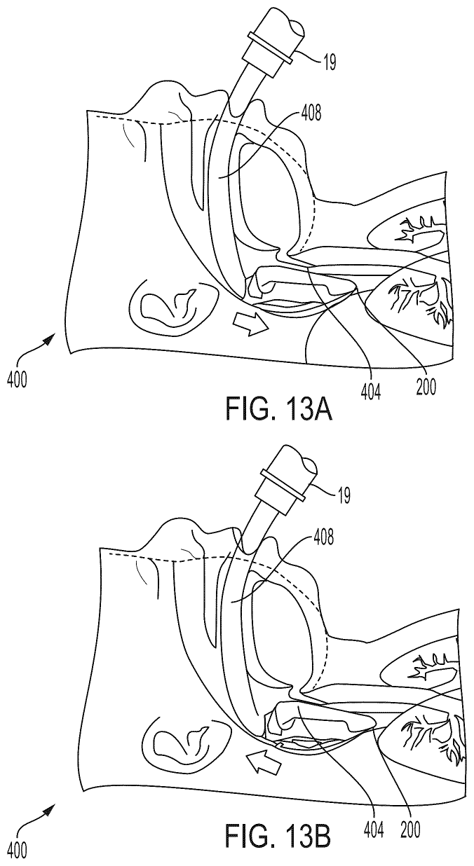

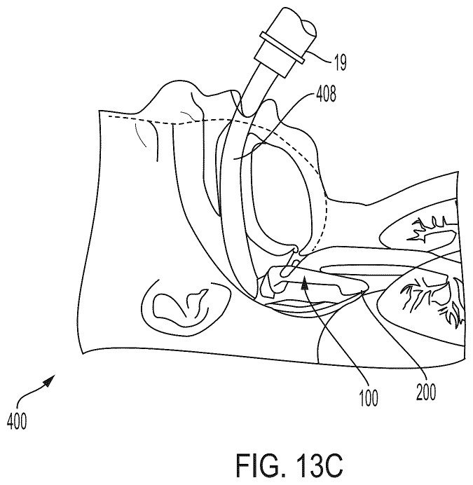

FIGS. 9-13C depict a method of inserting an AMD 10 in a patient 400 so that an airway can be provided to the patient 400. FIG. 9 depicts steps of such a method, and FIGS. 11-13C show the patient 400 as well as the laryngeal mask 13 during various stages of inserting the laryngeal mask 13 into the patient 400. In such a method, a medical services provider (such as a physician) may seek to position the EHRidge 99 (see also FIGS. 6A, 6F, 6G, 6H, 7A, 7C, 7E, 7F, and 7G) relative to the patient's epiglottis 404 so that a distal end 404A of the epiglottis 404 resides in the pocket 100 (see also FIGS. 6A, 6G, 6H, and 7C). In order to position the epiglottis 404 in the pocket 100, the laryngeal mask 13 may be provided and inserted 300 into the patient 400 and pushed 303 along the hard palate 408 (see FIG. 11) by pushing on the supply hub 19 so as to advance 306 the laryngeal mask 13 through the oropharynx into the hypopharynx (see FIG. 12) until resistance is met (caused by the hypopharynx pressing on the mask 13) and the distal tip 200 of the laryngeal mask 13 begins to enter 309 the patient's esophagus 412 (see FIG. 13A). Once the distal tip 200 has been pushed slightly further 309 into the esophagus 412, thereby allowing the EHRidge 99, and its crest 99A in particular, to slide beyond the epiglottis 404, the laryngeal mask 13 may be pulled back 312 (see FIG. 13B) so that the epiglottis 404 is held in the open-airway position (see FIG. 13C) by the EHRidge 99. In this position, the laryngeal mask 13 is in a neutral position in which it fits snugly without moving easily forward or backwards, unless a slight force is applied to the mask 13 via the delivery tube. In this manner, the glottis is open and available for gas to move 315 from the delivery tube to the patient's laryngeal inlet, and vice versa.

### A LIST OF FEATURE NUMBERS IS

10 AMD
13 laryngeal mask
16 delivery tube
17 in-use primary flow direction
19 supply hub
22 gas port
23 breathing gas source
31 face-region
32 cuff
34 anterior patient-contacting surface a.k.a. APCS
35 bowl
36A proximal end of the delivery tube
36B distal end of the delivery tube
37 DT gas channel
41 substantially flat surface of DT
43 radius of curvature
44 center of curvature
57 supply hub wall
78 hub gas channel
79 connector port
81 hub flange
82 annularly positioned surface of the flange
83 substantially flat portion of the annularly positioned surface
84 opening for receipt of DT
87A proximal side of mask
87B distal side of mask
90 anterior side of mask
96 ESTab
99 EP-holding ridge, a.k.a. EHRidge
99A Curved surface of the EHRidge
99B Crest of the curved surface 99A
100 EP-holding pocket
101 plane
105 posterior side of mask
108 posterior-side ridges
111 ridge height
114 left lateral-side-portion of cuff
117 right lateral-side-portion of cuff
120 ridge width
123 ridge length
126 side-grooves
127 lateral-side of bowl
129 lateral-side of the cuff
132 mask/DT connector
138A gas channel of the mask/DT connector
138B gas channel of the mask/DT connector
153A proximal end of supply hub
153B distal end of supply hub
156 seating-groove on flange
200 distal tip of mask
300 provide and insert mask
303 push mask along hard palate
306 push mask into hypopharynx
309 push mask until EHRidge is slightly beyond
312 pull mask back slightly
315 move air to/from mask and thereby the patient's laryngeal inlet
400 patient
404 epiglottis
404A distal end of epiglottis
408 hard palate of patient
412 esophagus

Now that features of the invention and some embodiments of the invention have been described, the following statements succinctly identify various embodiments of the invention, but the invention is not limited to these embodiments:

### A-Series Statements-EHPocket

Statement A1 (1). An airway management device (“AMD”), comprising:
a laryngeal mask having an anterior patient-contacting surface (“APCS”) shaped to contact a patient and at least partially surround the patient's laryngeal inlet when installed in the patient; and
a delivery tube having interior wall surfaces defining a gas channel (the “DT Gas Channel”), the DT Gas Channel being in gas-communication with a bowl of the laryngeal mask; and
an epiglottis-holding pocket that subtends part of the APCS, the epiglottis-holding pocket being bounded at least in part by the subtended part of the APCS, and wherein the epiglottis-holding pocket is positioned relative to the APCS so that, when the laryngeal mask is installed in a patient, at least a portion of an epiglottis of the patient resides in the epiglottis-holding pocket and is retracted away from the patient's laryngeal inlet.

Note: in some embodiments of the invention, the epiglottis-holding pocket is circumscribed by the APCS, or the anterior cuff, or both. In some embodiments of the invention, the epiglottis-holding pocket may extend from or include an area that is circumscribed by the APCS, or the anterior cuff, or both.

Statement A2 (2). The AMD of Statement A1 wherein the DT Gas Channel is configured to facilitate conveying gas:
(a) toward the bowl; and
(b) away from the bowl.

Statement A3 (3). The AMD of any one of Statements A1 or A2, wherein the laryngeal mask is not inflatable.

Statement A4 (4). The AMD of any one of Statement A1, A2, or A3, wherein the APCS has a Shore hardness of at least 5 and not more than 50 on the A-scale.

Statement A5 (5). The AMD of any one of Statements A1, A2, A3, or A4, wherein the laryngeal mask further comprises an epiglottis-holding ridge (“EHRidge”) that subtends a portion of the APCS, wherein the EHRidge is positioned relative to the APCS so that, when the laryngeal mask is installed in a patient, at least a portion of the EHRidge touches and bends the patient's epiglottis so that the epiglottis is retracted away from the patient's laryngeal inlet.

Note: In some embodiments of the invention, the EHRidge is circumscribed by the APCS, or the anterior cuff, or both. In some embodiments of the invention, the EHRidge may extend from an area that is circumscribed by the APCS, or the anterior cuff, or both.

Statement A6 (6). The AMD of Statement A5, wherein the EHRidge is curved.

Statement A7 (7). The AMD of any one of Statements A5 or A6, wherein a distal end of the delivery tube and the EHRidge are positioned so that a plane that is substantially perpendicular to an in-use primary flow direction of gas toward the bowl immediately prior to leaving the delivery tube intersects with:
(a) a distal end of the delivery tube; and
(b) the EHRidge.

Statement A8 (8). The AMD of any one of Statements A5, A6, or A7, wherein the epiglottis-holding pocket is formed at least in part by a surface of the EHRidge, and the epiglottis-holding pocket extends from the EHRidge toward the portion of the APCS subtended by the EHRidge.

Statement A9 (9). The AMD of any one of Statements A5, A6, A7, or A8, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

Statement A10 (10). The AMD any one of Statements A1 through A9, wherein the laryngeal mask has a plurality of ridges (the “posterior-side ridges”) on a posterior-side of the laryngeal mask opposite the APCS.

Statement A11 (11). The AMD of Statement A10, wherein the plurality of posterior-side ridges are substantially parallel to each other, and the posterior-side ridges are oriented in a direction that is substantially parallel to a primary direction in which the delivery tube extends.

Statement A12 (12). The AMD of any one of Statements A1 through A11, wherein the laryngeal mask has a first cuff-portion extending from the APCS to form a first lateral compression pocket adjacent to the bowl.

Statement A13 (13). The AMD of Statement A12, wherein the bowl extends between the delivery tube and the APCS, and the first lateral compression pocket is formed in part by a first lateral-side of the bowl.

Statement A14 (14). The AMD of Statement A13, wherein the laryngeal mask has a second cuff-portion extending from the APCS to form a second lateral compression pocket adjacent to a second lateral-side of the bowl.

Statement A15 (15). The AMD of any one of Statements A1 through A14, wherein the delivery tube has an exterior surface, a cross-section of which indicates a portion that is substantially flat, the cross-section being taken substantially perpendicular to an in-use primary flow direction of gas within the delivery tube.

Statement A16 (16). The AMD of any one of Statements A1 through A15, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

Statement A17 (17). The AMD of Statement A16, wherein the DT Gas Channels are configured to facilitate conveying gas toward and away from the bowl.

### B-Series Statements-EHRidge

Statement B1 (18). An airway management device (“AMD”), comprising:
a laryngeal mask having an anterior patient-contacting surface (“APCS”) shaped to contact a patient and at least partially surround the patient's laryngeal inlet when installed in the patient; and
a delivery tube having interior wall surfaces defining a gas channel (the “DT Gas Channel”), the DT Gas Channel being in gas-communication with a bowl of the laryngeal mask; and
an epiglottis-holding ridge (“EHRidge”) that subtends a portion of the APCS, wherein the EHRidge is positioned relative to the APCS so that, when the laryngeal mask is installed in a patient, at least a portion of the EHRidge touches and bends an epiglottis of the patient so that the epiglottis is retracted away from the patient's laryngeal inlet.

Note: In some embodiments of the invention, the EHRidge is circumscribed by the APCS, or the anterior cuff, or both. In some embodiments of the invention, the EHRidge may extend from an area that is circumscribed by the APCS, or the anterior cuff, or both.

Statement B2 (19). The AMD of Statement B1 wherein the DT Gas Channel is configured to facilitate conveying gas:
(a) toward the bowl; and
(b) away from the bowl.

Statement B3 (20). The AMD of any of Statements B1 or B2, wherein the laryngeal mask is not inflatable.

Statement B4 (21). The AMD of any one of Statements B1, B2, or B3, wherein the APCS has a Shore hardness of at least 5 and not more than 50 on the A-scale.

Statement B5 (22). The AMD of any one of Statements B1 through B4, wherein a distal end of the delivery tube and the EHRidge are positioned so that a plane that is substantially perpendicular to an in-use primary flow direction of gas toward the bowl immediately prior to leaving the delivery tube intersects with:
(a) a distal end of the delivery tube; and
(b) the EHRidge.

Statement B6 (23). The AMD of any one of Statements B1 through B5, wherein the EHRidge is curved.

Statement B7 (24). The AMD of any one of Statements B1 through B6, further comprising an epiglottis-holding pocket that subtends part of the APCS, the pocket being bounded at least in part by the subtended part of the APCS, and wherein the epiglottis-holding pocket is positioned relative to the APCS so that, when the laryngeal mask is installed in a patient, at least a portion of the patient's epiglottis resides in the pocket and is retracted away from the patient's laryngeal inlet.

Note: In some embodiments of the invention, the epiglottis-holding pocket is circumscribed by the APCS, or the anterior cuff, or both. In some embodiments of the invention, the epiglottis-holding pocket may extend from or include an area that is circumscribed by the APCS, or the anterior cuff, or both.

Statement B8 (25). The AMD of Statement B7, wherein the epiglottis-holding pocket is formed at least in part by a surface of the EHRidge, and the epiglottis-holding pocket extends from the EHRidge toward the portion of the APCS subtended by the EHRidge.

Statement B9 (26). The AMD of any one of Statements B7 or B8, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

Statement B10 (27). The AMD of any one of Statements B1 through B9, wherein the laryngeal mask has a plurality of ridges (the “posterior-side ridges”) on a posterior-side of the laryngeal mask opposite the APCS.

Statement B11 (28). The AMD of Statement B10, wherein the plurality of posterior-side ridges are substantially parallel to each other, and the posterior-side ridges are oriented in a direction that is substantially parallel to a primary direction in which the delivery tube extends.

Statement B12 (29). The AMD of any one of Statements B1 through B11, wherein the laryngeal mask has a first cuff-portion extending from the APCS to form a first lateral compression pocket adjacent to the bowl.

Statement B13 (30). The AMD of Statement B12, wherein the bowl extends between the delivery tube and the APCS, and the first lateral compression pocket is formed in part by a first lateral-side of the bowl.

Statement B14 (31). The AMD of Statement B12 or B13, wherein the laryngeal mask has a second cuff-portion extending from the APCS to form a second lateral compression pocket adjacent to a second lateral-side of the bowl.

Statement B15 (32). The AMD of any one of Statements B1 through B14, wherein the delivery tube has an exterior surface, a cross-section of which indicates a portion that is substantially flat, the cross-section being taken substantially perpendicular to an in-use primary flow direction of gas within the delivery tube.

Statement B16 (33). The AMD of any one of Statements B1 through B15, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

Statement B17 (34). The AMD of Statement B16, wherein the DT Gas Channels are configured to facilitate conveying gas toward and away from the bowl.

### C-Series Statements-Posterior-Side Ridges

Statement C1 (35). An airway management device (“AMD”), comprising:
a laryngeal mask having an anterior patient-contacting surface (“APCS”) shaped to contact a patient and at least partially surround the patient's laryngeal inlet when installed in the patient; and
a delivery tube having interior wall surfaces defining a gas channel (the “DT Gas Channel”), the DT Gas Channel being in gas-communication with a bowl of the laryngeal mask; and
a plurality of ridges (the “posterior-side ridges”) on a posterior-side of the laryngeal mask opposite the APCS;
wherein the plurality of posterior-side ridges are substantially parallel to each other, and the posterior-side ridges are oriented in a direction that is substantially parallel to a primary direction in which the delivery tube extends.

Statement C2 (36). The AMD of Statement C1 wherein the DT Gas Channel is configured to facilitate conveying gas:
(a) toward the bowl; and
(b) away from the bowl.

Statement C3 (37). The AMD of any one of Statements C1 or C2, wherein the laryngeal mask is not inflatable.

Statement C4 (38). The AMD of any one of Statements C1, C2, or C3, wherein the APCS has a Shore hardness of at least 5 and not more than 50 on the A-scale.

Statement C5 (39). The AMD of any one of Statements C1 through C4, wherein the laryngeal mask further comprises an epiglottis-holding ridge (“EHRidge”) that subtends a portion of the APCS, wherein the EHRidge is positioned relative to the APCS so that, when the laryngeal mask is installed in a patient, at least a portion of the EHRidge touches and bends an epiglottis of the patient so that the epiglottis is retracted away from the patient's laryngeal inlet.

Note: In some embodiments of the invention, the EHRidge is circumscribed by the APCS, or the anterior cuff, or both. In some embodiments of the invention, the EHRidge may extend from an area that is circumscribed by the APCS, or the anterior cuff, or both.

Statement C6 (40). The AMD of Statement C5, wherein the EHRidge is curved.

Statement C7 (41). The AMD of any one of Statements C5 or C6, wherein a distal end of the delivery tube and the EHRidge are positioned so that a plane that is substantially perpendicular to an in-use primary flow direction of gas toward the bowl immediately prior to leaving the delivery tube intersects with:
(a) a distal end of the delivery tube; and
(b) the EHRidge.

Statement C8 (42). The AMD of any one of Statements C1 through C7 further comprising an epiglottis-holding pocket that subtends a part of the APCS, the epiglottis-holding pocket being bounded at least in part by the subtended part of the APCS, and wherein the epiglottis-holding pocket is positioned relative to the APCS so that, when the laryngeal mask is installed in a patient, at least a portion of an epiglottis of the patient resides in the epiglottis-holding pocket and is retracted away from the patient's laryngeal inlet.

Note: In some embodiments of the invention, the epiglottis-holding pocket is circumscribed by the APCS, or the anterior cuff, or both. In some embodiments of the invention, the epiglottis-holding pocket may extend from or include an area that is circumscribed by the APCS, or the anterior cuff, or both.

Statement C9 (43). The AMD of Statement C8, wherein the epiglottis-holding pocket is formed at least in part by a surface of the EHRidge of Statement C5, C6, or C7, and the epiglottis-holding pocket extends from the EHRidge toward the portion of the APCS subtended by the epiglottis-holding pocket.

Statement C10 (44). The AMD of any one of Statements C8 or C9, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

Statement C11 (45). The AMD of any one of Statements C1 through C10, wherein the laryngeal mask has a first cuff-portion extending from the APCS to form a first lateral compression pocket adjacent to the bowl.

Statement C12 (46). The AMD of Statement C11, wherein the bowl extends between the delivery tube and the APCS, and the first lateral compression pocket is formed in part by a first lateral-side of the bowl.

Statement C13 (47). The AMD of Statement C11, wherein the laryngeal mask has a second cuff-portion extending from the APCS to form a second lateral compression pocket adjacent to a second lateral-side of the bowl.

Statement C14 (48). The AMD of any one of Statements C1 through C13, wherein the delivery tube has an exterior surface, a cross-section of which indicates a portion that is substantially flat, the cross-section being taken substantially perpendicular to an in-use primary flow direction of gas within the delivery tube.

Statement C15 (49). The AMD of any one of Statements C1 through C14, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

Statement C16 (50). The AMD of Statement C15, wherein the DT Gas Channels are configured to facilitate conveying gas toward and away from the bowl.

Statement C17 (51). The AMD of any one of Statements C1 through C16, wherein the posterior-side ridges are made from a material that will flex under forces expected to be exerted by a patient's pharynx when the mask resides in the patient.

Statement C18 (52). The AMD of any one of Statements C1 through C17, wherein the posterior-side ridges are made of a material having a shore hardness on the A-scale of at least 5 and not more than 50.

Statement C19 (53). The AMD of any one of Statements C1 through C18, wherein one or more of the posterior-side ridges have an aspect ratio (height: width) in a range from 0.53:1 to 22.38:1.

Statement C20 (54). The AMD of any one of Statements C1 through C19, wherein at least one of the posterior-side ridges is taller than another of the posterior-side ridges.

Statement C21 (55). The AMD of any one of Statements C1 through C20, wherein at least one of the posterior-side ridges does not have a constant height.

Statement C22 (56). The AMD of any one of Statements C1 through C21, wherein at least one of the posterior-side ridges does not have a constant width.

Statement C23 (57). The AMD of any one of Statements C1 through C22, wherein at least one of the posterior-side ridges is longer than another of the posterior-side ridges.

Statement C24 (58). The AMD of any one of Statements C1 through C23, wherein the bowl extends between the delivery tube and the APCS.

### D-Series Statements-Side Pockets/Grooves

Statement D1 (59). An airway management device (“AMD”), comprising:
a laryngeal mask having:
   (a) an anterior patient-contacting surface shaped to contact a patient and at least partially surround the patient's laryngeal inlet when installed in the patient;
   (b) a bowl positioned relative to the anterior patient-contacting surface to receive exhaled gas from the laryngeal inlet and provide breathing gas to the laryngeal inlet; and
   (c) a cuff extending from the anterior patient-contacting surface, the cuff having a lateral-side-portion extending from the anterior-patient-contacting surface and forming a lateral compression pocket adjacent to a lateral-side of the bowl.
a delivery tube having interior wall surfaces defining a gas channel (the “DT Gas Channel”), the DT Gas Channel being in gas-communication with the bowl of the laryngeal mask.

Statement D2 (60). The AMD of Statement D1 wherein the DT Gas Channel is configured to facilitate conveying gas:
(a) toward the bowl; and
(b) away from the bowl.

Statement D3 (61). The AMD of any one of Statements D1 or D2, wherein the laryngeal mask is not inflatable.

Statement D4 (62). The AMD of any one of Statements D1, D2, or D3, wherein the APCS has a Shore hardness of at least 5 and not more than 50 on the A-scale.

Statement D5 (63). The AMD of any one of Statements D1 through D4, wherein the laryngeal mask further comprises an epiglottis-holding ridge (“EHRidge”) that subtends a portion of the APCS, wherein the EHRidge is positioned relative to the APCS so that, when the laryngeal mask is installed in a patient, at least a portion of the EHRidge touches and bends an epiglottis of the patient so that the epiglottis is retracted away from the patient's laryngeal inlet.

Note: In some embodiments of the invention, the EHRidge is circumscribed by the APCS, or the anterior cuff, or both. In some embodiments of the invention, the EHRidge may extend from an area that is circumscribed by the APCS, or the anterior cuff, or both.

Statement D6 (64). The AMD of Statement D5, wherein the EHRidge is curved.

Statement D7 (65). The AMD of any one of Statements D5 or D6, wherein a distal end of the delivery tube and the EHRidge are positioned so that a plane that is substantially perpendicular to an in-use primary flow direction of gas toward the bowl immediately prior to leaving the delivery tube intersects with:
(a) a distal end of the delivery tube; and
(b) the EHRidge.

Statement D8 (66). The AMD of any one of Statements D1 through D7, further comprising an epiglottis-holding pocket that subtends a part of the APCS, the epiglottis-holding pocket being bounded at least in part by the subtended part of the APCS, and wherein the epiglottis-holding pocket is positioned relative to the APCS so that, when the laryngeal mask is installed in a patient, at least a portion of an epiglottis of the patient resides in the epiglottis-holding pocket and is retracted away from the patient's laryngeal inlet.

Note: In some embodiments of the invention, the epiglottis-holding pocket is circumscribed by the APCS, or the anterior cuff, or both. In some embodiments of the invention, the epiglottis-holding pocket may extend from or include an area that is circumscribed by the APCS, or the anterior cuff, or both.

Statement D9 (67). The AMD of Statement D8, wherein the epiglottis-holding pocket is formed at least in part by a surface of the EHRidge of Statement D5, D6 or D7, the EHRidge subtending a portion of the anterior-patient-contacting surface, and the epiglottis-holding pocket extends from the EHRidge toward the portion of the APCS subtended by the EHRidge.

Statement D10 (68). The AMD of Statement D8 or D9, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

Statement D11 (69). The AMD of any one of Statements D1 through D10, wherein the laryngeal mask has a plurality of ridges (the “posterior-side ridges”) on a posterior-side of the laryngeal mask opposite the APCS.

Statement D12 (70). The AMD of Statement D11, wherein the plurality of posterior-side ridges are substantially parallel to each other, and the posterior-side ridges are oriented in a direction that is substantially parallel to a primary direction in which the delivery tube extends.

Statement D13 (71). The AMD of any one of Statements D1 through D12, wherein the delivery tube has an exterior surface, a cross-section of which indicates a portion that is substantially flat, the cross-section being taken substantially perpendicular to an in-use primary flow direction of gas within the delivery tube.

Statement D14 (72). The AMD of any one of Statements D1 through D13, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

Statement D15 (73). The AMD of Statement D14, wherein the DT Gas Channels are configured to facilitate conveying gas toward and away from the bowl.

Statement D16 (74). The AMD of any one of Statements D1 through D15, wherein another lateral-side-portion of the cuff extends from the APCS, and is positioned to form another lateral compression pocket adjacent to a different lateral-side of the bowl.

Statement D17 (75). The AMD of any one of Statements D1 through D15, wherein the lateral compression pocket is oriented such that the compression pocket extends from a closed side of the compression pocket to an open side of the compression pocket in a direction that is away from the APCS.

Statement D18 (76). The AMD of any one of Statements D1 through D15, wherein the lateral compression pocket has an opening adjacent to a posterior-side of the mask.

Statement D19 (77). The AMD of any one of Statements D1 through D16, wherein the bowl extends between the delivery tube and the APCS, and the lateral compression pocket is formed in part by a lateral-side of the bowl.

Statement D20 (78). The AMD of Statements D18 or D19, wherein the laryngeal mask has a second cuff portion extending from the APCS to form a second lateral compression pocket adjacent to a second lateral-side of the bowl

Statement D21 (79). The AMD of Statement D1 or D20, wherein the lateral-side-portion(s) of the cuff is/are made from a material that permits the lateral-side-portion to flex toward the bowl when the AMD is inserted into a patient.

Although the present invention has been described with respect to one or more particular embodiments, it is to be understood that other embodiments of the present invention may be made without departing from the spirit and scope of the present invention. Hence, the present invention is deemed limited only by the appended claims and the reasonable interpretation thereof.

What is claimed is:

1. An airway management device (“AMD”), comprising:
a laryngeal mask having an anterior patient-contacting surface shaped to contact a patient and at least partially surround the patient's laryngeal inlet when installed in the patient; and
a delivery tube having interior wall surfaces defining a gas channel (the “DT Gas Channel”), the DT Gas Channel being in gas-communication with a bowl of the laryngeal mask; and
an epiglottis-holding pocket that subtends part of the anterior patient-contacting surface, the epiglottis-holding pocket being bounded at least in part by the subtended part of the anterior patient-contacting surface, and wherein the epiglottis-holding pocket is positioned relative to the anterior patient-contacting surface so that, when the laryngeal mask is installed in a patient, at least a portion of an epiglottis of the patient resides in the epiglottis-holding pocket and is retracted away from the patient's laryngeal inlet.

2. The AMD of claim 1 wherein the DT Gas Channel is configured to facilitate conveying gas:
(a) toward the bowl; and
(b) away from the bowl.

3. The AMD of claim 1, wherein the laryngeal mask is not inflatable.

4. The AMD of claim 1, wherein the anterior patient-contacting surface has a Shore hardness of at least 5 and not more than 50 on the A-scale.

5. The AMD of claim 1, wherein the laryngeal mask further comprises an epiglottis-holding ridge that subtends a portion of the anterior patient-contacting surface, wherein the epiglottis-holding ridge is positioned relative to the anterior patient-contacting surface so that, when the laryngeal mask is installed in a patient, at least a portion of the epiglottis-holding ridge touches and bends the patient's epiglottis so that the epiglottis is retracted away from the patient's laryngeal inlet.

6. The AMD of claim 5, wherein the epiglottis-holding ridge is curved.

7. The AMD of claim 5, wherein a distal end of the delivery tube and the epiglottis-holding ridge are positioned so that a plane that is substantially perpendicular to an in-use primary flow direction of gas toward the bowl immediately prior to leaving the delivery tube intersects with:
(a) a distal end of the delivery tube; and
(b) the epiglottis-holding ridge.

8. The AMD of claim 5, wherein the epiglottis-holding pocket is formed at least in part by a surface of the epiglottis-holding ridge, and the epiglottis-holding pocket extends from the epiglottis-holding ridge toward the portion of the anterior patient-contacting surface subtended by the epiglottis-holding ridge.

9. The AMD of claim 8, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

10. The AMD of claim 1, wherein the laryngeal mask has a plurality of ridges on a posterior-side (“posterior-side ridges”) of the laryngeal mask opposite the anterior patient-contacting surface.

11. The AMD of claim 10, wherein the plurality of posterior-side ridges are substantially parallel to each other, and the posterior-side ridges are oriented in a direction that is substantially parallel to a primary direction in which the delivery tube extends.

12. The AMD of claim 1, wherein the laryngeal mask has a first cuff-portion extending from the anterior patient-contacting surface to form a first lateral compression pocket adjacent to the bowl.

13. The AMD of claim 12, wherein the bowl extends between the delivery tube and the anterior patient-contacting surface, and the first lateral compression pocket is formed in part by a first lateral-side of the bowl.

14. The AMD of claim 13, wherein the laryngeal mask has a second cuff-portion extending from the anterior patient-contacting surface to form a second lateral compression pocket adjacent to a second lateral-side of the bowl.

15. The AMD of claim 1, wherein the delivery tube has an exterior surface, a cross-section of which indicates a portion that is substantially flat, the cross-section being taken substantially perpendicular to an in-use primary flow direction of gas within the delivery tube.

16. The AMD of claim 1, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

17. The AMD of claim 16, wherein the DT Gas Channels are configured to facilitate conveying gas toward and away from the bowl.

18. An airway management device (“AMD”), comprising:
a laryngeal mask having an anterior patient-contacting surface shaped to contact a patient and at least partially surround the patient's laryngeal inlet when installed in the patient; and
a delivery tube having interior wall surfaces defining a gas channel (the “DT Gas Channel”), the DT Gas Channel being in gas-communication with a bowl of the laryngeal mask; and
an epiglottis-holding ridge that subtends a portion of the anterior patient-contacting surface, wherein the epiglottis-holding ridge is positioned relative to the anterior patient-contacting surface so that, when the laryngeal mask is installed in a patient, at least a portion of the epiglottis-holding ridge touches and bends an epiglottis of the patient so that the epiglottis is retracted away from the patient's laryngeal inlet.

19. The AMD of claim 18 wherein the DT Gas Channel is configured to facilitate conveying gas:
(a) toward the bowl; and
(b) away from the bowl.

20. The AMD of claim 18, wherein the laryngeal mask is not inflatable.

21. The AMD of claim 18, wherein the anterior patient-contacting surface has a Shore hardness of at least 5 and not more than 50 on the A-scale.

22. The AMD of claim 18, wherein a distal end of the delivery tube and the epiglottis-holding ridge are positioned so that a plane that is substantially perpendicular to an in-use primary flow direction of gas toward the bowl immediately prior to leaving the delivery tube intersects with:
(a) a distal end of the delivery tube; and
(b) the epiglottis-holding ridge.

23. The AMD of claim 18, wherein the epiglottis-holding ridge is curved.

24. The AMD of claim 18, further comprising an epiglottis-holding pocket that subtends part of the anterior patient-contacting surface, the pocket being bounded at least in part by the subtended part of the anterior patient-contacting surface, and wherein the epiglottis-holding pocket is positioned relative to the anterior patient-contacting surface so that, when the laryngeal mask is installed in a patient, at least a portion of the patient's epiglottis resides in the pocket and is retracted away from the patient's laryngeal inlet.

25. The AMD of claim 24, wherein the epiglottis-holding pocket is formed at least in part by a surface of the epiglottis-holding ridge, and the epiglottis-holding pocket extends from the epiglottis-holding ridge toward the portion of the anterior patient-contacting surface subtended by the epiglottis-holding ridge.

26. The AMD of claim 24, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

27. The AMD of claim 18, wherein the laryngeal mask has a plurality of ridges on a posterior-side (“posterior-side ridges”) of the laryngeal mask opposite the anterior patient-contacting surface.

28. The AMD of claim 27, wherein the plurality of posterior-side ridges are substantially parallel to each other, and the posterior-side ridges are oriented in a direction that is substantially parallel to a primary direction in which the delivery tube extends.

29. The AMD of claim 18, wherein the laryngeal mask has a first cuff-portion extending from the anterior patient-contacting surface to form a first lateral compression pocket adjacent to the bowl.

30. The AMD of claim 29, wherein the bowl extends between the delivery tube and the anterior patient-contacting surface, and the first lateral compression pocket is formed in part by a first lateral-side of the bowl.

31. The AMD of claim 29, wherein the laryngeal mask has a second cuff-portion extending from the anterior patient-contacting surface to form a second lateral compression pocket adjacent to a second lateral-side of the bowl.

32. The AMD of claim 18, wherein the delivery tube has an exterior surface, a cross-section of which indicates a portion that is substantially flat, the cross-section being taken substantially perpendicular to an in-use primary flow direction of gas within the delivery tube.

33. The AMD of claim 18, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

34. The AMD of claim 33, wherein the DT Gas Channels are configured to facilitate conveying gas toward and away from the bowl.

35. An airway management device (“AMD”), comprising:
a laryngeal mask having an anterior patient-contacting surface shaped to contact a patient and at least partially surround the patient's laryngeal inlet when installed in the patient; and
a delivery tube having interior wall surfaces defining a gas channel (the “DT Gas Channel”), the DT Gas Channel being in gas-communication with a bowl of the laryngeal mask; and
a plurality of ridges (the “posterior-side ridges”) on a posterior-side of the laryngeal mask opposite the anterior patient-contacting surface;
wherein the plurality of posterior-side ridges are substantially parallel to each other, and the posterior-side ridges are oriented in a direction that is substantially parallel to a primary direction in which the delivery tube extends.

36. The AMD of claim 35 wherein the DT Gas Channel is configured to facilitate conveying gas:
(a) toward the bowl; and
(b) away from the bowl.

37. The AMD of claim 35, wherein the laryngeal mask is not inflatable.

38. The AMD of claim 35, wherein the anterior patient-contacting surface has a Shore hardness of at least 5 and not more than 50 on the A-scale.

39. The AMD of claim 35, wherein the laryngeal mask further comprises an epiglottis-holding ridge that subtends a portion of the anterior patient-contacting surface, wherein the epiglottis-holding ridge is positioned relative to the anterior patient-contacting surface so that, when the laryngeal mask is installed in a patient, at least a portion of the epiglottis-holding ridge touches and bends an epiglottis of the patient so that the epiglottis is retracted away from the patient's laryngeal inlet.

40. The AMD of claim 39, wherein the epiglottis-holding ridge is curved.

41. The AMD of claim 39, wherein a distal end of the delivery tube and the epiglottis-holding ridge are positioned so that a plane that is substantially perpendicular to an in-use primary flow direction of gas toward the bowl immediately prior to leaving the delivery tube intersects with:
(a) a distal end of the delivery tube; and
(b) the epiglottis-holding ridge.

42. The AMD of claim 35 further comprising an epiglottis-holding pocket that subtends a part of the anterior patient-contacting surface, the epiglottis-holding pocket being bounded at least in part by the subtended part of the anterior patient-contacting surface, and wherein the epiglottis-holding pocket is positioned relative to the anterior patient-contacting surface so that, when the laryngeal mask is installed in a patient, at least a portion of an epiglottis of the patient resides in the epiglottis-holding pocket and is retracted away from the patient's laryngeal inlet.

43. The AMD of claim 42, wherein the epiglottis-bolding pocket is formed at least in part by a surface of an epiglottis-holding ridge, and the epiglottis-holding pocket extends from the epiglottis-holding ridge toward the portion of the anterior patient-contacting surface subtended by the epiglottis-holding pocket.

44. The AMD of claim 42, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

45. The AMD of claim 35, wherein the laryngeal mask has a first cuff-portion extending from the anterior patient-contacting surface to form a first lateral compression pocket adjacent to the bowl.

46. The AMD of claim 45, wherein the bowl extends between the delivery tube and the anterior patient-contacting surface, and the first lateral compression pocket is formed in part by a first lateral-side of the bowl.

47. The AMD of claim 45, wherein the laryngeal mask has a second cuff-portion extending from the anterior patient-contacting surface to form a second lateral compression pocket adjacent to a second lateral-side of the bowl.

48. The AMD of claim 35, wherein the delivery tube has an exterior surface, a cross-section of which indicates a portion that is substantially flat, the cross-section being taken substantially perpendicular to an in-use primary flow direction of gas within the delivery tube.

49. The AMD of claim 35, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

50. The AMD of claim 49, wherein the DT Gas Channels are configured to facilitate conveying gas toward and away from the bowl.

51. The AMD of claim 35, wherein the posterior-side ridges are made from a material that will flex under forces expected to be exerted by a patient's pharynx when the mask resides in the patient.

52. The AMD of claim 51, wherein the posterior-side ridges are made of a material having a shore hardness on the A-scale of at least 5 and not more than 50.

53. The AMD of claim 35, wherein one or more of the posterior-side ridges have an aspect ratio (height: width) in a range from 0.53:1 to 22.38:1.

54. The AMD of claim 35, wherein at least one of the posterior-side ridges is taller than another of the posterior-side ridges.

55. The AMD of claim 35, wherein at least one of the posterior-side ridges does not have a constant height.

56. The AMD of claim 35, wherein at least one of the posterior-side ridges does not have a constant width.

57. The AMD of claim 35, wherein at least one of the posterior-side ridges is longer than another of the posterior-side ridges.

58. The AMD of claim 35, wherein the bowl extends between the delivery tube and the anterior patient-contacting surface.

59. An airway management device (“AMD”), comprising:
a laryngeal mask having:
(a) an anterior patient-contacting surface shaped to contact a patient and at least partially surround the patient's laryngeal inlet when installed in the patient;
(b) a bowl positioned relative to the anterior patient-contacting surface to receive exhaled gas from the laryngeal inlet and provide breathing gas to the laryngeal inlet; and
(c) a cuff extending from the anterior patient-contacting surface, the cuff having a first lateral-side-portion extending from the anterior patient-contacting surface and forming a first lateral compression pocket adjacent to a lateral-side of the bowl,
a delivery tube having interior wall surfaces defining a gas channel (the “DT Gas Channel”′), the DT Gas Channel being in gas-communication with the bowl of the laryngeal mask.

60. The AMD of claim 59 wherein the DT Gas Channel is configured to facilitate conveying gas:
(a) toward the bowl; and
(b) away from the bowl.

61. The AMD of claim 59, wherein the laryngeal mask is not inflatable.

62. The AMD of claim 59, wherein the anterior patient-contacting surface has a Shore hardness of at least 5 and not more than 50 on the A-scale.

63. The AMD of claim 59, wherein the laryngeal mask further comprises an epiglottis-holding ridge that subtends a portion of the anterior patient-contacting surface, wherein the epiglottis-holding ridge is positioned relative to the anterior patient-contacting surface so that, when the laryngeal mask is installed in a patient, at least a portion of the epiglottis-holding ridge touches and bends an epiglottis of the patient so that the epiglottis is retracted away from the patient's laryngeal inlet.

64. The AMD of claim 63, wherein the epiglottis-holding ridge is curved.

65. The AMD of claim 63, wherein a distal end of the delivery tube and the epiglottis-holding ridge are positioned so that a plane that is substantially perpendicular to an in-use primary flow direction of gas toward the bowl immediately prior to leaving the delivery tube intersects with:
(a) a distal end of the delivery tube; and
(b) the epiglottis-holding ridge.

66. The AMD of claim 59, further comprising an epiglottis-holding pocket that subtends a part of the anterior patient-contacting surface, the epiglottis-holding pocket being bounded at least in part by the subtended part of the anterior patient-contacting surface, and wherein the epiglottis-holding pocket is positioned relative to the anterior patient-contacting surface so that, when the laryngeal mask is installed in a patient, at least a portion of an epiglottis of the patient resides in the epiglottis-holding pocket and is retracted away from the patient's laryngeal inlet.

67. The AMD of claim 66, wherein the epiglottis-holding pocket is formed at least in part by a surface of an epiglottis-holding ridge, the epiglottis-holding ridge subtending a portion of the anterior-patient-contacting surface, and the epiglottis-holding pocket extends from the epiglottis-holding ridge toward the portion of the anterior patient-contacting surface subtended by the epiglottis-holding ridge.

68. The AMD of claim 66, wherein a distal end of the delivery tube and the epiglottis-holding pocket are positioned so that a plane that is substantially perpendicular to an in-use primary flow direction of gas toward the bowl immediately prior to leaving the delivery tube intersects with:
(a) a distal end of the delivery tube; and
(b) the epiglottis-holding pocket.

69. The AMD of claim 59, wherein the laryngeal mask has a plurality of ridges (“posterior-side ridges”) on a posterior-side of the laryngeal mask opposite the anterior patient-contacting surface.

70. The AMD of claim 69, wherein the plurality of posterior-side ridges are substantially parallel to each other, and the posterior-side ridges are oriented in a direction that is substantially parallel to a primary direction in which the delivery tube extends.

71. The AMD of claim 59, wherein the delivery tube has an exterior surface, a cross-section of which indicates a portion that is substantially flat, the cross-section being taken substantially perpendicular to an in-use primary flow direction of gas within the delivery tube.

72. The AMD of claim 59, wherein the interior wall surfaces of the delivery tube define at least two gas channels (the “DT Gas Channels”), wherein:
(a) a first one of the DT Gas Channels (the “First DT Gas Channel”) is configured to facilitate gas-communication with the bowl, but not directly with a second one of the DT Gas Channels (the “Second DT Gas Channel”); and
(b) the Second DT Gas Channel is configured to facilitate gas-communication with the bowl, but not directly with the First DT Gas Channel.

73. The AMD of claim 72, wherein the DT Gas Channels are configured to facilitate conveying gas toward and away from the bowl.

74. The AMD of claim 59, wherein a second lateral-side-portion of the cuff extends from the anterior patient-contacting surface, and is positioned to form a second lateral compression pocket adjacent to a different lateral-side of the bowl.

75. The AMD of claim 59, wherein the lateral compression pocket is oriented such that the compression pocket extends from a closed side of the compression pocket to an open side of the compression pocket in a direction that is away from the anterior patient-contacting surface.

76. The AMD of claim 59, wherein the lateral compression pocket has an opening adjacent to a posterior-side of the mask.

77. The AMD of claim 59, wherein the bowl extends between the delivery tube and the anterior patient-contacting surface, and the first lateral compression pocket is formed in part by a lateral-side of the bowl.

78. The AMD of claim 59, wherein the lateral-side-portion of the cuff is made from a material that permits the lateral-side-portion to flex toward the bowl when the AMD is inserted into a patient.
